(19) United States
(12) Patent Application Publication
(10) Pub. No.: US 2025/0152053 A1
(43) Pub. Date: May 15, 2025

(54) PCLED LIGHT SOURCE AND SWIR SPECTROMETER FOR NONINVASIVE TISSUE GLUCOSE SELF-MONITORING

(71) Applicant: LUMILEDS LLC, San Jose, CA (US)

(72) Inventor: Peter Josef SCHMIDT, Aachen (DE)

(73) Assignee: LUMILEDS LLC, San Jose, CA (US)

(21) Appl. No.: 19/020,080

(22) Filed: Jan. 14, 2025

Related U.S. Application Data
(63) Continuation of application No. PCT/US2023/027900, filed on Jul. 17, 2023.
(60) Provisional application No. 63/396,705, filed on Aug. 10, 2022.

(51) Int. Cl. A61B 5/1455; A61B 5/145

(52) U.S. Cl. CPC A61B 5/1455; A61B 5/14532; A61B 2562/0233

(57) ABSTRACT

A glucose measurement device comprising a light emitting device comprising an SWIR phosphor having emission wavelengths in the range of 1600-2200 nm, the SWIR phosphor comprising a structurally disordered garnet material, a sensitizer ion, and at least one rare earth emitter ion, and a infrared light detector arranged to detect the intensity of short wavelength infrared light emitted by the light emitting device and reflected by a sample. The emission spectra provided by the light emitting device having a high temperature stability at infrared absorption minima and maxima wavelengths of glucose in tissue.

### CROSS-REFERENCE TO RELATED APPLICATIONS

This application is a continuation of International Application No. PCT/US2023/027900 filed Jul. 17, 2023, which claims benefit of priority to U.S. Provisional Patent Application No. 63/396,705 titled “PCLED LIGHT SOURCE AND SWIR SPECTROMETER FOR NONINVASIVE TISSUE GLUCOSE SELF-MONITORING” and filed Aug. 10, 2022. Each of the forementioned applications is incorporated herein by reference in its entirety.

This application is related to U.S. Provisional Patent Application No. 63/235,523 filed Aug. 20, 2021, which is incorporated herein by reference in its entirety.

### FIELD OF THE INVENTION

The disclosure relates generally to noninvasive sensing devices for monitoring biological molecules using short wave infrared emitting phosphor-converted light-emitting devices, and in particular, the disclosure relates to glucose sensors that include phosphor compositions for use in phosphor-converted light-emitting devices, and more particularly to glucose sensors incorporating phosphor compositions having broadband infrared emission in the 1600-2200 nm wavelength range and high temperature stability.

### BACKGROUND

Semiconductor light emitting diodes and laser diodes (collectively referred to herein as “LEDs”) are among the most efficient light sources currently available. The emission spectrum of an LED typically exhibits a single narrow peak at a wavelength determined by the structure of the device and by the composition of the semiconductor materials from which it is constructed. By suitable choice of device structure and material system, LEDs may be designed to operate at ultraviolet, visible, or infrared wavelengths.

LEDs may be combined with one or more wavelength converting materials (generally referred to herein as “phosphors”) that absorb light emitted by the LED and in response emit light of a longer wavelength. For such phosphor-converted LEDs (“pcLEDs”), the fraction of the light emitted by the LED that is absorbed by the phosphors depends on the amount of phosphor material in the optical path of the light emitted by the LED, for example on the concentration of phosphor material in a phosphor layer disposed on or around the LED and the thickness of the layer.

Phosphor-converted LEDs may be designed so that all of the light emitted by the LED is absorbed by one or more phosphors, in which case the emission from the pcLED is entirely from the phosphors. In such cases the phosphor may be selected, for example, to emit light in a spectral region that is not efficiently generated directly by an LED.

Alternatively, pcLEDs may be designed so that only a portion of the light emitted by the LED is absorbed by the phosphors, in which case the emission from the pcLED is a mixture of light emitted by the LED and light emitted by the phosphors. By suitable choice of LED, phosphors, and phosphor composition, such a pcLED may be designed to emit, for example, light having a desired color temperature and desired color-rendering properties.

### SUMMARY

In one aspect, a glucose measurement device is provided, the glucose measuring device including a light emitting device comprising an SWIR phosphor having emission wavelengths in the range of 1600-2200 nm, the SWIR phosphor comprising a structurally disordered garnet material, a sensitizer ion, and at least one rare earth emitter ion; and an infrared light detector arranged to detect the intensity of short wavelength infrared light emitted by the light emitting device and reflected by a sample. The at least one rare earth emitter ion may include Ho (III). The sensitizer ion may include Cr(III), and the at least one rare earth emitter ion may include Tm(III) and Ho(III). The SWIR phosphor may include (Gd_{3-u-v-x-y-z}LuₓTm_{y}Ho_{z}ScᵥREᵤ)[Sc_{2-a-b-d-e}LuₐCr_{b}Ga_{d}Alₑ]{Ga_{3-c}Al_{c}}O₁₂ with RE=La, Y, Yb, Nd, Er, Ce and 0<u<2,0<v≤1 0 x≤1,0<y≤0.5,0<z≤0.05,0<a≤1,0<b≤0.3,0<c≤3,0<d≤1.8,0<e≤1.8. TheSWIR phosphor may include Gd₂O₃- 0.0065 Ho₂O₃-0.1 Tm₂O₃-0.33 Sc₂O₃-0.12 Lu₂O₃-0.8 Ga₂O₃-0.04 Cr₂O₃- 1.6 Al₂O₃. The SWIR phosphor may be formed into a ceramic plate, the ceramic plate may include (Al,Ga)₂O₃ as a minority phase. The light emitting device may include an InGaN primary light source, the SWIR phosphor may be formed into a wavelength converting structure disposed on a light emitting face of the primary light source, the wavelength converting structure may further include at least two alternating silica and niobia oxide layers disposed on a surface of the wavelength converting structure opposite the primary light source. The light emitting device may include a light emitting surface, the light emitting surface having an area of 1 cm×1 cm or less. The light emitting device may include an array of pcLEDs. The infrared light detector may include a filter element, the filter element may be configured to selectively pass at least one of the wavelength ranges at a glucose infrared absorption maxima and a glucose infrared absorption minima. The infrared light detector may include a filter element configured to selectively pass at least one of the wavelength ranges 1942±0-12 nm, 2098±0-12 nm, 1890±0-12 nm, and 2004±0-12 nm. The light emitting device may be configured to emit a SWIR spectral power output of >15 mW when driven at or near 150 mA. The light emitting device may be configured to emit a spectrum that is thermally stable in the range of human skin temperatures. The light emitting device is configured to emit a spectrum that has less than a 0.1±0.02% /K linear intensity change at the glucose absorption spectrum maxima and minima over the temperature range 26° C.-56° C. The light emitting device may be positioned adjacent the infrared light detector, a light emitting surface of the light emitting device facing the sample, and a light receiving surface of the infrared light detector facing the sample. The glucose measurement device may further include a processor connected to the light emitting device and the infrared light detector.

In another aspect, a smartphone including the glucose measurement device is provided.

In yet another aspect, a wearable device including the glucose measurement device is provided.

In yet another aspect, a handheld device including the glucose measurement device is provided.

In yet another aspect, a method for determining glucose levels in a tissue sample comprising measuring reflection spectra from the skin sample at at least one of the wavelengths within intervals 1942±0-12 nm, 2098±0-12 nm, 1890±0-12 nm, and 2004±0-12 nm using the glucose measurement.

### BRIEF DESCRIPTION OF THE DRAWINGS

FIG. 1 illustrates an embodiment of a wavelength converting structure as part of an infrared light-emitting device.

FIG. 2 illustrates another embodiment of a wavelength converting structure as part of an infrared light-emitting device.

FIG. 3 is a cross-section view of one example of an LED.

FIG. 4 is a cross sectional view of an IR emitting device with a wavelength converting structure in direct contact with an LED.

FIG. 5 is a cross sectional view of an IR emitting device with a wavelength converting structure in close proximity to an LED.

FIG. 6 is a cross sectional view of an IR emitting device with a wavelength converting structure spaced apart from an LED.

FIGS. 7A and 7B illustrate IR spectrometers including an SWIR light emitting devices with sensors positioned for, respectively, absorption and reflection spectroscopy. FIG. 7C illustrates a top plan view of a light emitting surface of an SWIR light emitting device in IR spectrometers.

FIGS. 8A and 8B show, respectively, cross-sectional and top schematic views of an array of SWIR pcLEDs.

FIG. 9A shows a schematic top view of an electronics board on which an array of pcLEDs may be mounted, and FIG. 9B similarly shows an array of SWIR pcLEDs mounted on the electronic board of FIG. 9A.

FIG. 10A shows a schematic cross-sectional view of an array of SWIR pcLEDs arranged with respect to waveguides and a projection lens. FIG. 10B shows an arrangement similar to that of FIG. 10A, without the waveguides.

FIG. 11 schematically illustrates an example camera flash system comprising an adaptive illumination system.

FIG. 12 schematically illustrates an example display (e.g., AR/VR/MR) system that includes an adaptive illumination system.

FIG. 13 shows the X-ray powder pattern (copper radiation) of an Gd_{2.367}Ho_{0.01}Tm_{0.152}Sc_{1.6}Lu_{0.27}Ga_{1.8}Al_{1.78}Cr_{0.04}O₁₂ SWIR phosphor.

FIG. 14 shows a scanning electron microscopy (SEM) image of an Gd_{2.367}Ho_{0.01}Tm_{0.152}Sc_{1.6}Lu_{0.27}Ga_{1.8}Al_{1.78}Cr_{0.04}O₁₂ SWIR phosphor.

FIG. 15 shows a power reflectance spectrum of an Gd_{2.367}Ho_{0.01}Tm_{0.152}Sc_{1.6}Lu_{0.27}Ga_{1.8}Al_{1.78}Cr_{0.04}O₁₂ SWIR phosphor in the visible spectral range.

FIG. 16 shows the normalized short-wave infrared emission spectrum of an SWIR pcLED including an Gd_{2.367}Ho_{0.01}Tm_{0.152}Sc_{1.6}Lu_{0.27}Ga_{1.8}Al_{1.78}Cr_{0.04}O₁₂ SWIR phosphor.

FIG. 17 shows the X-ray powder pattern (copper radiation) of Gd_{2.59}Tm_{0.24}Ho_{0.02}Sc_{0.75}Lu_{0.3}Ga₂Al₂Cr_{0.1}O₁₂ SWIR phosphor.

FIG. 18 shows the normalized short-wave infrared emission spectrum of the SWIR pcLED including an Gd_{2.59}Tm_{0.24}Ho_{0.02}Sc_{0.75}Lu_{0.3}Ga₂Al₂Cr_{0.1}O₁₂ SWIR phosphor.

FIG. 19A shows a scanning electron micrograph of the sintered SWIR phosphor ceramic of Example 5a. FIG. 19B shows a scanning electron micrograph of the sintered SWIR phosphor ceramic of Example 5b.

FIG. 20 shows the light transmission of the coated ceramic plate of Example 6a.

FIG. 21 SWIR emission spectrum of the phosphor converted LED of Example 7.

FIG. 22A schematically illustrates an IR spectroscopy test apparatus. FIG. 22B schematically illustrates the IR spectroscopy test apparatus of FIG. 22A with a test sample.

FIG. 23 is the FT-IR spectrum of a polystyrene test sample.

FIG. 24 is the spectral power distribution in the SWIR wavelength range of the phosphors of Example 8.

FIG. 25 shows the SWIR emission spectrums of the SWIR light emitting device of Example 9 at three different temperatures.

FIG. 26 shows the intensity change of the SWIR light emitting device of Example 9 at four selected wavelength intervals suitable for tissue glucose sensing as a function of the light source temperature over a range of human skin temperatures.

### DETAILED DESCRIPTION

The following detailed description should be read with reference to the drawings, in which identical reference numbers refer to like elements throughout the different figures. The drawings, which are not necessarily to scale, depict selective embodiments and are not intended to limit the scope of the invention. The detailed description illustrates by way of example, not by way of limitation, the principles of the invention.

Diabetes mellitus (“diabetes”) refers to a group of diseases that affect how the body uses blood sugar (“glucose”). Diabetes is typically treated by injecting insulin into the patient in amounts needed to stabilize blood glucose levels within acceptable ranges. Long-term complications of diabetes develop gradually. The longer a patient has diabetes, and the less control a patient has of their blood glucose levels, the higher the risk of developing complications from diabetes. Eventually, diabetes complications may be disabling or even life-threatening. Thus, tight control of blood glucose levels via intensive insulin therapy in diabetes patients can significantly delay many serious complications. Such intensive insulin therapy requires careful monitoring of a patient's blood glucose concentrations via frequent glucose measurements, to track blood glucose levels, and adjustment of insulin dosages based on the measured glucose levels. As such, a convenient method and device for measuring glucose is useful for the better management of diabetes.

Current glucose monitoring methods may include either a finger prick with a lancet to obtain a blood sample, or use of a continuous monitoring device which requires insertion of a sensor wire under the skin. As glucose monitoring needs to be done often, methods that pierce the skin may be painful and/or cause irritation, and thus create difficulty with treatment compliance. A device for non-invasive glucose monitoring that does not require piercing the skin is therefore desirable. Furthermore, it is desirable that such a device be wearable, and thus extremely compact, so as not to interfere with daily activities.

In non-patent literature Heise, H.M.; Delbeck, S.; Marbach, R. Noninvasive Monitoring of Glucose Using Near-Infrared Reflection Spectroscopy of Skin Constraints and Effective Novel Strategy in Multivariate Calibration. Biosensors 2021, 11, 64 (https://doi.org/10.3390/bios11030064) (referred to herein as Heise et al. and incorporated herein by reference) describe a method for non-invasive monitoring of blood glucose. The method allows noninvasive glucose monitoring by means of reflectance FTIR spectroscopy of the tissue of the inner lower lip. A multivariate, science-based calibration method is used to obtain usable glucose level information from skin tissue diffuse reflection SWIR spectroscopy. According to the authors, a sufficiently large contribution of the glucose analyte signal to the overall spectrum that is well above observed spectral noise is required to unambiguously detect glucose within the complex matrix provided by the skin tissue. The device used in Heise to measure glucose, is, however, large and unwieldly, and would be extremely inconvenient for a patient to routinely incorporate into their regular daily activities.

Thus, for the multivariate calibration method described by Heise et al. to be used in real life applications by diabetes patent, a glucose sensing device is needed that has much more compact and efficient light sources and sensing technologies, to enable improved signal to noise ratios, shorter measurement times and eventually enable system integration of light source and detector into, e.g., wearable devices. In addition to being compact, such glucose sensing device needs to provide stable readings in various conditions when being worn, and in particular, provide stable readings over the varying range of potential body temperatures of the patient.

As noted in Heise, when measuring glucose through human tissue, the maxima wavelengths of glucose absorption occur at approximately 1942 nm and 2098 nm and the minima wavelengths of glucose absorption occurs approximately 1890 nm and 2004 nm. As such, glucose sensing devices using IR spectra of glucose through tissue require light emitting devices providing significant power output and temperature stability over infrared radiation in the short wavelength ranges.

This specification discloses phosphors for use in light emitting devices that may be particularly useful for measuring tissue glucose, and incorporation of those phosphors into light sources for miniature glucose monitors. Glucose levels measured through tissue non-invasively are directly related to blood glucose levels and thus are useful for monitoring glucose and treating diabetes. In particular, the SWIR phosphor disclosed herein have emission spectra covering the range of the tissue glucose absorption spectra, have relatively high emissions output, and are temperature stable over the range of human body temperatures.

Phosphors disclosed herein can emit infrared (“IR”) radiation in the short wavelength infrared radiation range (“SWIR”), more specifically, SWIR phosphors that can emit infrared radiation having a peak wavelength in a range of 1600 nm to 2200 nm. In particular, the SWIR phosphors disclosed herein are capable of providing a continuous emission spectrum, without emission gaps, while maintaining a high conversion efficiency, in the range of 1600 nm to 2200 nm. For example, SWIR phosphors disclosed herein may provide a continuous emission spectrum over a spectral width of at least 500 nm, and have a minimum spectral power in the 1600 nm to 2200 nm emission range that is at least 20% of the median spectral power in this range. Such SWIR phosphor, emitting with good spectral power in 1600 nm to 2200 nm wavelength range, are especially useful for measurement of tissue glucose, as the emission range encompasses both the tissue glucose absorption maxima (around 1942 nm and 2098 nm) and minima (around1890 nm and 2004 nm).

SWIR phosphors disclosed herein may be excited by light with wavelengths in the blue spectral range. The broad-band SWIR phosphor materials can efficiently convert shorter wavelength blue light into broad-band emission with longer SWIR wavelengths. For economy of language, infrared radiation may be referred to herein as “infrared light,” “IR light,” or “light.”

The specification also discloses light sources that include the SWIR phosphor disclosed herein. Such light sources may include a primary light source, such as an LED, and a wavelength converting structure that includes an SWIR phosphor that emits wavelengths in the 1600-2200 nm range as disclosed herein, to form, for example, an SWIR pcLED. Use of broad-band SWIR phosphors in such light sources may extend the wavelengths emitted by the light source to up to 2200 nm, while providing a continuous spectral power distribution over a wide wavelength range and maintaining a high conversion efficiency.

The wavelength converting structure in the light source may further include additional phosphors. The additional phosphor may include, for example, other SWIR phosphors that emit wavelengths in other portions of the infrared wavelength range, for example, into the 1000-1700 nm spectral range. The additional phosphors may also allow for more efficient excitation of the SWIR phosphors.

This specification also discloses wavelength converting structure devices that includes such broad-band SWIR phosphors. The wavelength converting structure devices having the broad-band SWIR phosphors may further include additional phosphors.

The specification also discloses devices useful for non-invasive measurement of tissue glucose levels. In particular, the specification discloses use of light emitting devices having the broad-band SWIR phosphors in a spectrometer device used for IR absorption and reflection spectroscopy applications. The light emitting devices in these IR spectrometers have an SWIR phosphor, such as an SWIR pcLED as disclosed herein, instead of traditional incandescent light sources, such as tungsten filament lamps. Currently, tungsten filament light sources that have a CCT in the range of 2300K are being used to cover the spectral range needed for SWIR spectrometer systems (e.g., 1000 nm to 3000 pm). The tungsten filament light sources, however, lack the mechanical robustness, fast modulating capabilities to increase spectrometer sensitivity, and compactness that is required for, for example, IR spectroscopy integration into small hand held devices, and wearable devices or smartphones. And furthermore lack the longevity required for devices used frequently and in real life situations. The SWIR phosphors disclosed herein combine the broadened emission bands as preferred for spectroscopy applications with the high conversion efficiency, which means that they require less power, and can be used in miniaturized devices, such as wearable devices, and in particular wearable devices useful for glucose monitoring.

Furthermore, light sources disclosed herein may produce an emission spectrum that is extremely temperature stable, especially over the range of human body temperatures that a wearable device would experience positioned on a human.

The disclosed light sources are efficient and allow further miniaturization and cost reduction of sensors that cover the 1600-2200 nm wavelength range, as well as the 1000 nm to 2200 nm range.

### SWIR Phosphor Compositions

SWIR phosphor compounds disclosed herein include (i) a structurally disordered garnet host lattice material; (ii) at least one sensitizer ion; and (iii) at least one rare earth emitter ion.

A structurally disordered garnet host lattice material is a garnet lattice that has multiple chemically different doping sites. The disordered structure of the garnet lattice provides a multinary host that broadens the emission bands of the rare earth emitter ion dopant, resulting in a broad-band emission spectrum, but also maintains enough crystalline structure to provide high conversion efficiency. As used herein, the term “structurally disordered” refers to a material that possesses an ordered average structure or long-range translational periodicity that can be, e.g., characterized with an X-ray diffraction experiment. The different crystallographic lattice sites are however populated by chemically different atom species in a non-ordered but more statistical way, leading to a large variety of chemically slightly different substitutional lattice sites for the sensitizer and emitter ions, and thus to inhomogeneously broadened spectral features which is highly desired for the application of the SWIR phosphors. The host lattice has a substantial influence on the optical properties of a dopant, and variations of its chemical surroundings cause variations in the crystal fields at the dopant site, which can result in inhomogeneous, and hence, broader, emission. Therefore, use of a structurally disordered garnet host lattice as disclosed herein can help provide a more continuous emissions spectrum. At the same time, use of a host lattice that is too disordered may cause a drop in conversion efficiency due to, e.g., high concentrations of optically active lattice defects. In particular, the wanted broadening effect here for Cr3+ sensitized converters is realized by relatively high amounts of added elements like Lu, Sc and Al into the garnet material

Structurally disordered garnet host lattice compositions used herein may be derived from gadolinium gallium garnet Gd₃Ga₂Ga₃O₁₂ with three 8-fold coordinated Gd atoms, two 6-fold coordinated Ga atoms, and three 4-fold coordinated Ga atoms. Example host lattice compositions may include atoms that can occupy more than one lattice site, such as, for example, Lu and Sc (which have possible 8- and 6-fold coordination) and Al (which has possible 6- and 4-fold coordination). The disorder is caused by a statistical distribution of one sort of element over various lattice sites of the garnet structure. For example, Lutetium or Scandium may occupy the 8-fold and 6-fold coordinated lattice sites while Gallium and Aluminum can occupy the 6-fold and 4-fold coordinated lattice sites in concentrations that exceed that of trace or defect levels (>1 atom %). If the host lattice is further being doped with, e.g., Chromium(III) and Thulium according to (Gd,Lu,Sc,Tm)₃[Sc,Lu,Ga,Al,Cr]₂{Ga,Al}₃O₁₂, these dopants occupy multiple chemically different 6-fold or 8-fold coordinated sites respectively caused by the statistical distribution of the multiple site occupying host lattice elements. As a consequence, this disorder may lead to the desired broadening of e.g. absorption and emission transitions of Chromium(III) and Thulium, respectively. The mixed occupation of the sites lead to multiple different emitting sites in terms of oxygen ligand charge and distance and/or coordination geometry. Such structurally disordered structures lead to a broadening of composed emission bands and thus to a more even distribution of the spectral power over the desired range.

An example of a structurally disordered garnet host lattice is (Gd,Lu,Sc)₃(Sc,Lu, Ga)₂(Ga,Al)₃O₁₂ crystallizing in the cubic garnet structure type. In this example the composition of the binary oxides forming the garnet phase is chosen as such that lutetium is incorporated on the 8-fold and 6-fold coordinated cation sites. Alternatively, another example of a structurally disordered host lattice is (Gd,Lu,Sc)₃Sc₂(Ga,Al)₃O₁₂, which also crystallizes in the cubic garnet structure type, and has Sc atoms on both 8-fold and 6-fold coordinated sites.

SWIR phosphors disclosed herein are doped with at least one sensitizer ion. The sensitizer ion efficiently absorbs blue or red pump light, for instance from an LED, and transfers the absorbed energy to rare earth emitter ions that eventually emit in the desired spectral ranges. To efficiently absorb excitation light from a primary LED light source in the blue or red spectral range, the host material may be doped for example with Cr(III) as the sensitizer ion on the 6-fold coordinated sites.

SWIR phosphors disclosed herein are doped with at least one rare earth emitter ion, or a combination of rare earth emitter ions, that provide emission in the desired spectral range. For example, an SWIR phosphor that provides emission in the 1600-2200 nm wavelength range may have the 8-fold coordinated sites doped with Tm(III) and Ho(III) or with Tm(III) only. In addition, the host lattice can be doped with Er(III) to extend the emission range towards −1500 nm. The excitation and emission properties can be further tuned by replacing an additional part of Gd by La, Y, Yb, Nd, or Ce.

SWIR phosphors disclosed herein may have compositions that include phosphors from the class of garnet materials having a composition of: (Gd_{3-u-v-x-y-z}LuₓTm_{y}Ho_{z}ScᵥREᵤ)[Sc_{2-a-b-d-e}LuₐCr_{b}Ga_{d} Alₑ]{Ga_{3-c}Al_{c}}O₁₂ with RE=La, Y, Yb, Nd, Er, Ce and 0<u<2, 0<v≤1, 0<x<1, 0<y≤0.5, O z≤0.05, 0<a≤1, 0<b≤0.3, 0<c 3, 0<d≤1.8, 0<e≤1.8. Examples of SWIR phosphor having compositions in this class of garnet materials are described in more detail below, and include Gd_{2.367}Ho_{0.01}Tm_{0.152}Sc_{1.6}Lu_{0.27}Ga_{1.8}Al_{1.78}Cr_{0.04}O₁₂, Gd_{2.59}Tm_{0.24}Ho_{0.02}Sc_{0.75}Lu_{0.3}Ga₂Al₂Cr_{0.1}O₁₂, Gd₂Ho_{0.013}Tm_{0.2}Sc_{0.67}Lu_{0.24}Ga_{1.6}Al_{3.2}Cr_{0.08}O₁₂, and Gd_{2.67}Ho_{0.01}Tm_{0.17}Sc_{1.8}Lu_{0.3}Ga₂AlCr_{0.05}O₁₂.

To enhance the crystal quality of the SWIR phosphors, fluxing agents such as fluorides can be applied during manufacturing of the SWIR phosphor compositions, e.g., powder phosphor processing, which may result in elements from such fluxing agents being incorporated into the SWIR composition. An example of a flux system useful for the SWIR phosphor compositions is gadolinium fluoride. The application of fluoride fluxes may lead to the incorporation of some fluoride ions in the final SWIR phosphor composition without deteriorating its desired properties. An alternative flux system may be, for example, barium fluoride, BaF₂, or AlF₃ hydrate. Other example flux systems that may be applied include, for example silicon oxide, which may be useful in the manufacturing of ceramic phosphor wavelength converting structures, described in more detail below, that may be characterized as being polycrystalline, sintered luminescence converter elements that include the SWIR phosphors disclosed herein at least as part of the polycrystalline matrix. The silica fluxing agent may be added as fine silica powder or e.g. in form of a precursor such as an alkoxide like tetraethylorthosilicate that is being hydrolyzed during processing. Other parts of the polycrystalline matrix may be e.g. oxides such as aluminum oxide Al₂O₃ or mixed oxides like (Al,Ga)₂O₃.

Other compounds may be added to the SWIR phosphor composition if the amounts added are low enough so that the desired properties of the resulting SWIR phosphor are not greatly deteriorated but may lead to benefits like improved crystallization speed or better densification. Examples for such other compounds are, for example: alkaline earth compounds such as MgO, CaO or SrO, or the respective carbonates, zirconium or hafnium oxide, niobium or tantalum oxide, germanium oxide, silicon dioxide or other rare earth oxides not explicitly mentioned in the list above.

IR Light Emitting Devices having Wavelength Converting Structures Emitting over the 1600-2200 nm Wavelength Range

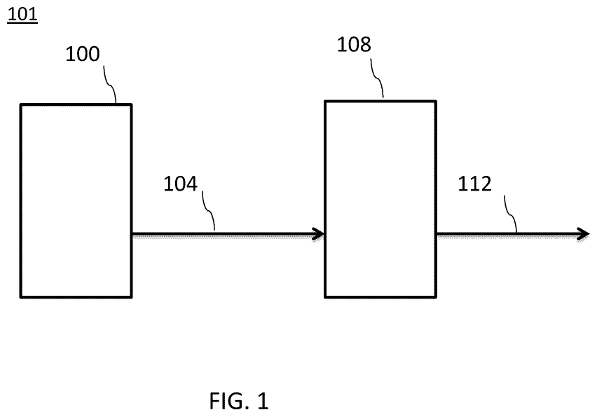

FIG. 1 illustrates an embodiment of an IR light emitting device that emits IR light over the 1600-2200 nm wavelength range. The IR light emitting device 101 includes a wavelength converting structure 108. Wavelength converting structure 108 includes at least one of the disclosed SWIR phosphors that emit in the 1600-2200 nm wavelength range. In addition to wavelength converting structure 108, illumination device 101 includes primary light source 100. The primary light source 100 may be an LED or any other suitable source including, as examples, resonant cavity light emitting diodes (RCLEDs) and vertical cavity laser diodes (VCSELs). For example, primary light source 100 may be a blue light emitting LED, or may include a red light emitting LED. Primary light source 100 emits a first light 104. A portion of the first light 104 is incident upon wavelength converting structure 108. The wavelength converting structure 108 absorbs the first light 104 and emits second light 112. The wavelength converting structure 108 may be structured such that little or no first light is part of the final emission spectrum from the device, though this is not required.

Wavelength converting structure 108 may include, for example, any of the SWIR phosphors disclosed here, such SWIR phosphors including a structurally disordered garnet host lattice, at least one sensitizer ion, and at least one rare earth emitter ion. For example, wavelength converting structure 108 may include an SWIR phosphor including a structurally disordered garnet host lattice, and Cr(III) sensitizer ion, and Tm(III) and Ho(III) rare earth emitter ions. For example, the wavelength converting structure 108 may include an SWIR phosphor from a class of garnet materials having a composition of: (Gd_{3-u-v-x-y-z}LuₓTm_{y}Ho_{z}ScᵥREᵤ)[Sc_{2-a-b-d-e}LuₐCr_{b}Ga_{d} Alₑ]{Ga_{3-c}Al_{c}}O₁₂ with RE=La, Y, Yb, Nd, Er, Ce and 0<u<2, 0<v≤1, 0<x<1, 0<y≤0.5, O z≤0.05, 0<a≤1, 0<b≤0.3, 0<c 3, 0<d≤1.8, 0<e≤1.8.

The wavelength converting structure 108 may include an SWIR phosphor that can be excited, for example, in the blue spectral range. For example, light source 100 may be an AlInGaN and/or an InGaN type emitter, and may emit first light 104 in the 440-460 nm wavelength range. The light source 100 may also be a light source that emit first light 104 in the red spectral range, for example, light source 100 may be an AlInGaP type emitter emitting wavelengths in the 600-650 nm wavelength range, or may be an InGaAs type emitter emitting wavelengths in the 700-1000 nm range, however, these red light emitting light sources may be less efficient at exciting the SWIR phosphor than light sources that emit in the blue wavelength range.

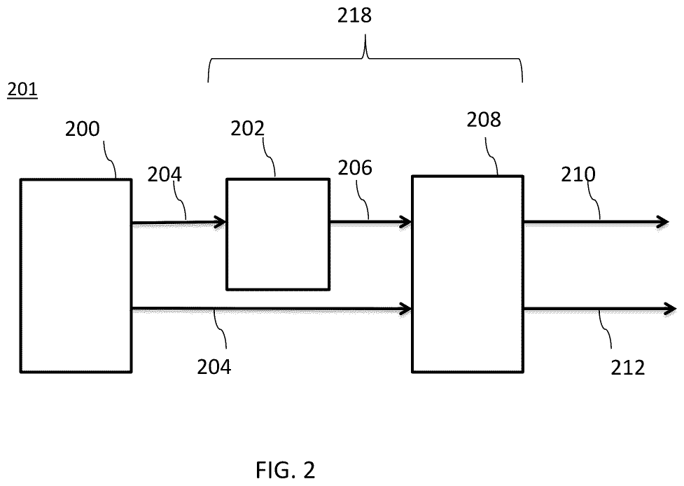

To improve the conversion efficiency of the SWIR phosphor included in wavelength converting structure 108, an additional phosphor, such as red emitting phosphor that can be excited by a blue emitting primary LED light source, may be included. FIG. 2 illustrates an IR light emitting device 201 in which a wavelength converting structure including one or more of the disclosed SWIR phosphor materials may further be combined with a second phosphor system. In FIG. 2, the wavelength converting structure 218 includes an SWIR phosphor portion 208 including the SWIR phosphors emitting in the 1600-2200 nm range as disclosed herein, and a second phosphor portion 202, as part of IR light-emitting device 201. In FIG. 2, a primary light source 200 may be an LED or any other suitable source, (including as examples resonant cavity light emitting diodes (RCLEDs) and vertical cavity laser diodes (VCSELs). Primary light source 200 emits first light 204.

First light 204 is incident upon wavelength converting structure 218, which includes an SWIR phosphor portion 208 including one or more of the SWIR phosphors disclosed herein, and a second phosphor system 202. A portion of the first light 204 is incident on a second phosphor portion 202 of the wavelength converting structure 218. The second phosphor 202 absorbs the first light 204 and emits third light 206. The third light 206 may have a wavelength range that is within the excitation range of the SWIR phosphor in the SWIR phosphor portion 208 of the wavelength converting structure 218. The third light 206 is incident on the SWIR phosphor portion 208. The SWIR phosphor portion 208 absorbs all or a portion of the third light 206 and emits fourth light 210. Additionally, a portion of the first light 204 may be incident on an SWIR phosphor portion 208 of the wavelength converting structure 218. The SWIR phosphor portion 208 may absorb the first light 204 and emit second light 212, or first light 204 may pass through the SWIR phosphor portion 208.

The wavelength converting structure 218 including an SWIR phosphor 208 and second phosphor 202 may be structured such that little or no first light or third light is part of the final emission spectrum from the device, though this is not required.

Examples of such a second phosphor system which may be useful for use in IR light-emitting device 201 include those disclosed in U.S. patent application Ser. No. 16/393,428 filed Sep. 13, 2018 and titled “Infrared Emitting Device” and incorporated herein by reference in its entirety. In particular, second phosphor 202 may be, for example, an Eu²⁺ doped red emitting material such as BSSNE type phosphors of composition M₂₋ₓSi_{5-y}Al_{y}O_{y}N_{8-y}:Euₓ (M=Ba, Sr, Ca), such as, for example Ba_{0.2}Ca_{0.06}Sr_{1.64}Si_{4.98}Al_{0.02}O_{0.02}N_{7.98}:Eu_{0.1}; CASN or SCASN type phosphors of composition M₁₋ₓSiAlN₃:Euₓ (M=Sr, Ca) such as, for example Ca_{.985}SiAlN₃:Eu_{0.015}; or M₁₋ₓLiAl₃N₄:Euₓ (M=Ba, Sr, Ca) such as, for example, (Ba_{0.5}Ca_{0.5}) _{0.995}LiAl₃N₄:Eu_{0.005}; or M₁₋ₓLi₂Al_{2-y}Si_{y}O₂-_{y}N₂₊_{y}:Euₓ (M=Ba, Sr, Ca) such as, for example, Sr_{0.996}Li₂Al_{1.996}Si_{0.004} O_{1.996}N_{2.004}:Eu_{0.004} which may crystallize in an ordered structure variant of the UCr₄C₄ structure type with Ba and Ca occupying specific lattice sites. Similar ordered variants are known for oxides like RbNaLi₆Si₂O₈. In (Ba_{0.5}Ca_{0.5})₁₋ₓLiAl₃N₄:Euₓ narrow band emission at ˜630 nm is obtained for Eu on Ba sites while NIR emission at wavelengths >700 nm is obtained for Eu on Ca sites. In other example, second phosphor 202 may be a CASN type phosphor of composition Ca_{0.985}SiAlN:Eu_{0.015}. CASN type red emitting phosphors are commercially available from e.g. Mitsubishi Chemical (BR-101 series).

The SWIR phosphor disclosed herein can be further combined with dielectric coating structures, for example by using an alternating SiO₂ and Nb₂O₅ layers having thicknesses in the range of 40 nm - 140 nm. A dichroic coating that reflects primary pump LED light and transmits the phosphor emitted light in the SWIR range can be a useful solution to enhance the performance of the IR light emitting devices. That is, such a dichroic coating may back-reflect blue light that then gets a chance to be reabsorbed by the wavelength converting structure, without changing the emission spectrum provided by the SWIR phosphor.

In FIG. 2, although the wavelength converting structure 218 is shown with the SWIR phosphor 208 and second phosphor system 202 as two separated blocks, in other embodiments, the wavelength converting structure the SWIR phosphor 208 and second phosphor system 202 may be combined or mixed. Methods of forming wavelength converting structures 108 and 218 are described in more detail below.

IR Light Emitting Devices having Wavelength Converting Structures Emitting over the 1100-2200 nm Wavelength Range

The SWIR phosphors disclosed herein can be further combined with additional IR phosphors to widen the wavelength range of IR emission emitted from the IR light emitting device. For example, the SWIR phosphors disclosed herein may be combined with additional IR phosphors that emit IR light at shorter wavelengths below the 1600-2200 nm wavelength range of the SWIR phosphors disclosed herein, to expand the wavelength range of light emitted by the IR light emitting device into shorter wavelengths.

Referring again to FIG. 1, the wavelength converting structure 108 may include, for example, the SWIR phosphors disclosed herein and additional IR phosphors. Primary light source 100 emits a first light 104. A portion of the first light 104 is incident upon wavelength converting structure 108 that includes, in this example, one or more additional IR phosphor in addition to the one or more SWIR phosphors disclosed herein. The wavelength converting structure 108 absorbs the first light 104 and emits second light 112. Because the wavelength converting structure 108 includes both the SWIR phosphor and the additional IR phosphor, the second light 112 emits IR light over a wide wavelength range that includes that of the additional IR phosphor as well as the SWIR phosphor 1600-2200 nm disclosed herein.

In one example, the additional IR phosphors included are shorter wavelength IR emitting phosphors disclosed in U.S. patent application Ser. No. 17/035,233, filed Sep. 23, 2020, titled “SWIR pcLED and Phosphor Emitting in the 1100-1700 nm Range,” incorporated herein by reference in its entirety. In particular, the additional IR phosphor may be one or more Niz+, or Ni²⁺ and Cr³⁺ doped spinel, perovskite, and garnet type IR phosphor emitting in the 1000-1700 nm range. For example, the additional IR phosphor may include Li_{0.5-0.5x}(Ga,Sc)_{2.5-0.5x-y}O₄:Nix,Cr_{y} (where 0<x<1, 0<y 0. 1, 0<z≤1, 0 K u<0.2) spinel type additional IR phosphor, and device 101 may include in the primary light source 100 a 620-630 nm emitting AlInGaP type LED. More specifically, device 101 may have a wavelength converting structure 108 that includes Li_{0.49}Sc_{0.05}Ga_{2.384}O₄:Ni_{0.013},Cr_{0.05} spinel type additional IR phosphor, and may include as primary light source 100 a 622 nm emitting AlInGaP type LED. Combining such additional IR phosphors with the SWIR phosphors disclosed herein extends the IR emission range of the device 101 to wavelengths shorter than the 1600 nm - 2200 nm range, so that the lighting device 101 emits in the 1100 nm - 2200 nm.

In another example, the additional IR phosphor included may also need an additional second phosphor system, similar to the second phosphor system described above for use with the SWIR phosphors, in the IR light emitting device having a wider IR emission range. Referring again to FIG. 2, the second phosphor system for use with the additional IR phosphor can widen the spectral range that allows efficient excitation of the additional IR phosphor, and thus increase number of the types of primary light sources 200 that may be used in device 201. That is, the additional second phosphor system for use with the additional IR phosphor 202 may be included with or without the second phosphor system disclosed above for use with the SWIR phosphors disclosed herein. The additional second phosphor system that absorbs first light 204 from a primary light source 200 that emits light outside of the wavelength range required to excite the additional IR phosphor. For instance, the additional second phosphor system 202 may absorb first light 204 emitted from a blue or green LED as primary light source 200. The second phosphor system 202 then emits third light 206 in the red spectral range. The third light 206 emitted from second phosphor system 202 excites the additional IR phosphor portion 208.

For example, device 201 may include green to red emitting phosphors, such as Eu²⁺ phosphors, added as the additional second phosphor system 202, and may use a blue emitting LED as the primary light source 200. Examples of a red emitting phosphor for use in additional second phosphor system 202 include (Sr,Ca)AlSiN₃:Eu and (Ba,Sr,Ca)₂Si₅₋ₓAlₓOₓN₈₋ₓ:Eu.

In an example device, primary light source 200 may be a blue light emitting InGaN type emitter. The wavelength converting structure 218 may include an orange-red emitting (Ba,Sr)₂Si₅N₈:Eu phosphor as the additional second phosphor system 202 and a Li_{0.5-0.5x}(Ga,Sc)_{2.5-0.5x-y}O₄:Nix,Cr_{y} spinel phosphor as the additional IR phosphor portion 212 as well as an SWIR phosphor as disclosed herein. In particular, device 201 may include as a primary light source 200 a 440-460 nm emitting InGaN type emitter, and a wavelength converting structure 218 that includes orange-red emitting phosphor (Ba_{0.4}Sr_{0.6})₂₋ₓSi₅N₈:Eu_{0.02} as the additional second phosphor system and a Li_{0.49}Sc_{0.05}Ga_{2.384}O₄:Ni_{0.013},Cr_{0.05} as the additional IR phosphor, as well as the SWIR phosphor disclosed herein. The additional second phosphor system 202 may include Cr³⁺ doped phosphors that emit in the 700-1000 nm wavelength range and that can be excited in the blue to green and red spectral ranges. The emission light of such Cr3+ phosphors being reabsorbed by Ni²⁺ doped additional IR phosphors. The additional second phosphor system 202 may include other Ni²⁺ phosphor systems that are known from the literature. Examples include LaMgGa₁₁O₁₉:Ni, MgO:Ni, MgF₂:Ni, Ga₂O₃:Ni,Ge, or garnets of composition RE₂AEMg₂TV₃O₁₂:Ni (RE=Y, La, Lu, Gd, Nd, Yb, Tm, Er; AE=Ca, Sr; TV=Si, Ge).

IR Light Emitting Devices having Wavelength Converting Structures with Temperature Stable Emissions at Glucose Absorption Maxima and Minima

SWIR phosphors useful for the detection of glucose may include Ho(III) as a rare earth emitter ion co-dopant with emission ranges over the 1600 nm - 2000 nm range, encompassing the tissue glucose wavelengths at both absorption maximas (approximately 1942 nm and 2098 nm) and both absorption minimas (approximately 1890 nm and 2004 nm). Such SWIR phosphors may additionally include Tm(III) as an additional rare earth emitter ion and Cr(III) as sensitizer. Such SWIR phosphors have a highly stable emissions spectrum (wavelength and temperature stability) and are especially suited for use in IR light emitting devices that can be incorporated into IR spectrometers used for non-invasive detection of tissue glucose.

While not wishing to be bound by any particular theory, the inner shell transition of the Ho(III) emitting ion allows such Ho(III)-containing SWIR phosphors to have (i) high spectral stability; (ii) predictive temperature dependence; and (iii) high conversion efficiency. These properties provide an advantage for sensing tissue glucose, as they allow the tiny tissue glucose signals to be detected within the large and varying background signals of the tissue. Thus, light sources that include such Ho(III)-containing SWIR phosphors are especially suitable for application in wearable tissue glucose measurement devices.

Such SWIR phosphors may include a Cr(III) sensitizer to absorb blue primary light, a Tm(III) rare earth ion emitter (sometimes referred to as an activator) and the Ho(III) rare earth ion emitter (again sometimes referred to as an activator) that shows wavelength and intensity stable emission in the tissue glucose absorption band wavelength ranges. As disclosed above, derivatives of gadolinium gallium garnets are examples of host lattices in which to incorporate these doping atoms. Such host lattices have, e.g., compositions: (Gd_{3-u-v-x-y-z}LuₓTm_{y}Ho_{z}ScᵥREᵤ)[Sc_{2-a-b-d-e}LuₐCr_{b}Ga_{d} Alₑ]{Ga_{3-c}Al_{c}}O₁₂ with RE=La, Y, Yb, Nd, Er, Ce and 0<u<2, 0<v≤1, 0<x<1, 0<y≤0.5, 0<z≤0.05, 0<a≤1, 0<b≤0.3, 0<c 3, 0<d≤1.8, 0<e≤1.8. An SWIR phosphor useful for application in tissue glucose measurement devices, and in particular wearable tissue glucose measurement devices, may, for example, have the composition 1.0 Gd₂O₃- 0.0065 Ho₂O₃-0.1 Tm₂O₃-0.33 Sc₂O₃-0.12 Lu₂O₃-0.8 Ga₂O₃- 0.04 Cr₂O₃- 1.6 Al₂O₃. In another example, an SWIR phosphor useful for applications in tissue glucose measurement devices, and in particular wearable tissue glucose measurement devices, may have the composition 1.405 Gd₂O₃- 0.005 Ho₂O₃-0.09 Tm₂O₃-1.35 Ga₂O₃- 0.05 Cr₂O₃- 1.15 Al₂O₃.

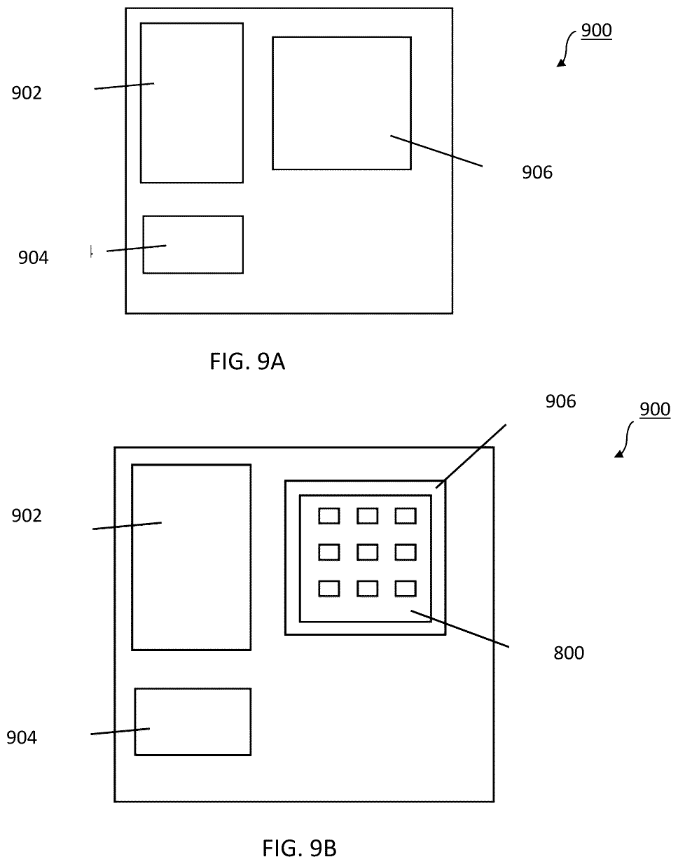

A wavelength converting structure, for example wavelength converting structure 108 of FIG. 1 or 218 of FIG. 2, (or phosphor pixels 806 of array 800 in FIG. 8A) may be formed using the SWIR phosphors including the Ho(III) activator. The SWIR phosphor may be formed into a ceramic, such as a polycrystalline ceramic plate. The polycrystalline ceramic may only contain one crystalline phase or more than one crystalline phase to form such a composite ceramic. In one example, the main phase includes a luminescent garnet phase with a composition as described in the formula above and an additional alumina phase, as will be described in more detail in the examples below. The additional alumina phase improves sinterability and optical properties of the luminescent composite ceramic.

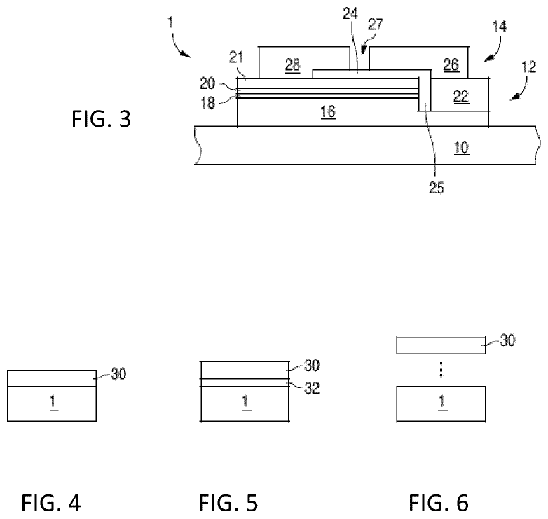

To form an IR light emitting device useful for detection of glucose, the wavelength converting structure may be formed using the SWIR phosphor that includes the Ho(III) activator, may be attached to a primary light source (for example, primary light sources 100, 200 of FIGS. 1 and 2) as described in more detail with respect to FIGS. 3, 4, 5, and 6. The primary light source may be an InGaN blue emitting primary light source. Additionally the IR emitting light source may further include a light scattering side coating, and a dichroic top coating deposited on the emitting surface of the wavelength converting element.

In particular, a realized example of such an IR light emitting device useful for detection of glucose may include a garnet ceramic phosphor as described above attached to a blue emitting InGaN primary LED (for example, an InGaN light emitting diode having a LUXEON® Rubix type architecture (Lumileds Holdings B.V.)). As shown in the below examples, such IR light emitting devices have a high SWIR output power (for example, >15 mW at 150 mA DC) and also have a very thermally stable spectrum (for example, −0.1±0.02%/K linear intensity change for the spectral intervals around the glucose absorption spectra minima and maxima over the ambient temperature range of 26-56° C.).

LEDs for Use with SWIR Phosphors

As shown in FIGS. 1 and 2, an IR light emitting device may include a wavelength converting structure that may be used, for example, with primary light source 100, 200. Primary light source 100, 200 may be a light emitting diode (LED). Light emitted by the light emitting diode is absorbed by the phosphors in the wavelength converting structure according to embodiments and emitted at a different wavelength. FIG. 3 illustrates one example of a suitable light emitting diode, a III-nitride LED that emits blue light for use in an IR light emitting device.

Though in the example below the semiconductor light emitting device is a III-nitride LED that emits blue or UV light, semiconductor light emitting devices besides LEDs such as laser diodes and semiconductor light emitting devices made from other materials systems such as other III-V materials, III-phosphide, Ill-arsenide, II-VI materials, ZnO, or Si-based materials may be used, as determined by, for example, the range of wavelengths needed to excite the SWIR phosphor, or combination of SWIR phosphor and second phosphor, in the wavelength converting structure.

FIG. 3 illustrates a III-nitride LED 1 that may be used in embodiments of the present disclosure. Any suitable semiconductor light emitting device may be used and embodiments of the disclosure are not limited to the device illustrated in FIG. 3. The device of FIG. 3 is formed by growing a III-nitride semiconductor structure on a growth substrate 10 as is known in the art. The growth substrate is often sapphire but may be any suitable substrate such as, for example, SiC, Si, GaN, or a composite substrate. A surface of the growth substrate on which the III-nitride semiconductor structure is grown may be patterned, roughened, or textured before growth, which may improve light extraction from the device. A surface of the growth substrate opposite the growth surface (i.e. the surface through which a majority of light is extracted in a flip chip configuration) may be patterned, roughened or textured before or after growth, which may improve light extraction from the device.

The semiconductor structure includes a light emitting or active region sandwiched between n- and p-type regions. An n-type region 16 may be grown first and may include multiple layers of different compositions and dopant concentration including, for example, preparation layers such as buffer layers or nucleation layers, and/or layers designed to facilitate removal of the growth substrate, which may be n-type or not intentionally doped, and n- or even p-type device layers designed for particular optical, material, or electrical properties desirable for the light emitting region to efficiently emit light. A light emitting or active region 18 is grown over the n-type region. Examples of suitable light emitting regions include a single thick or thin light emitting layer, or a multiple quantum well light emitting region including multiple thin or thick light emitting layers separated by barrier layers. A p-type region 20 may then be grown over the light emitting region. Like the n-type region, the p-type region may include multiple layers of different composition, thickness, and dopant concentration, including layers that are not intentionally doped, or n-type layers.

After growth, a p-contact is formed on the surface of the p-type region. The p-contact 21 often includes multiple conductive layers such as a reflective metal and a guard metal which may prevent or reduce electromigration of the reflective metal. The reflective metal is often silver but any suitable material or materials may be used. After forming the p-contact 21, a portion of the p-contact 21, the p-type region 20, and the active region 18 is removed to expose a portion of the n-type region 16 on which an n-contact 22 is formed. The n- and p-contacts 22 and 21 are electrically isolated from each other by a gap 25 which may be filled with a dielectric such as an oxide of silicon or any other suitable material. Multiple n-contact vias may be formed; the n- and p-contacts 22 and 21 are not limited to the arrangement illustrated in FIG. 3. The n- and p-contacts may be redistributed to form bond pads with a dielectric/metal stack, as is known in the art.

In order to form electrical connections to the LED 1, one or more interconnects 26 and 28 are formed on or electrically connected to the n- and p-contacts 22 and 21. Interconnect 26 is electrically connected to n-contact 22 in FIG. 3. Interconnect 28 is electrically connected to p-contact 21. Interconnects 26 and 28 are electrically isolated from the n- and p-contacts 22 and 21 and from each other by dielectric layer 24 and gap 27. Interconnects 26 and 28 may be, for example, solder, stud bumps, gold layers, or any other suitable structure.

The substrate 10 may be thinned or entirely removed. In some embodiments, the surface of substrate 10 exposed by thinning is patterned, textured, or roughened to improve light extraction.

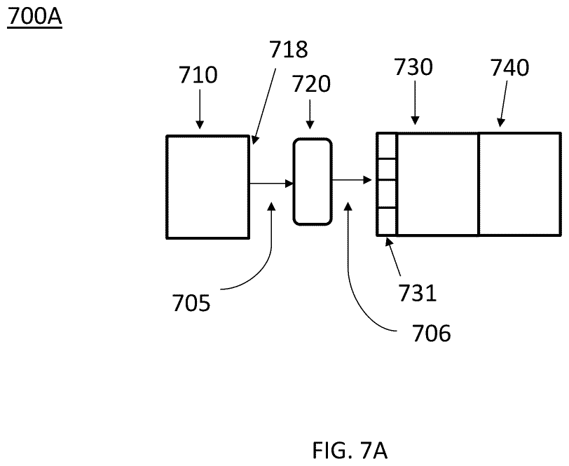

Any suitable light emitting device may be used in light sources according to embodiments of the disclosure. The invention is not limited to the particular LED illustrated in FIG. 3. The light source, such as, for example, the LED illustrated in FIG. 3, is illustrated in the following FIGS. 4, 5 and 6 by block 1.

Formation of SWIR Phosphors and Wavelength Converting Structures Including SWIR Phosphors

SWIR phosphors disclosed herein can be formed using any suitable method. In one example method, stable compounds, such as, for example, oxides, containing the elements to be formed into the garnet host, sensitizer ion, and rare earth element are mixed in appropriate ratios by, for example, ball milling. The mixture may then be fired at high temperatures, e.g., over 1500 C, with intermediate ball milling. The obtained powder may then be washed, for example, with water, dried, and sieved to form a powder of the SWIR phosphor material having particles with diameters in the range determined by the sieve, for example, less than 50 pm when a 50 pm sieve is used. The resulting SWIR phosphor powder is then used to form wavelength converting structures as described herein.

The wavelength converting structure, such as for example 108 described with respect to FIG. 1 or 218 described with respect to FIG. 2, which may contain one or more of the SWIR phosphors, or a combination of one or more of the SWIR phosphors and one or more of the additional IR phosphors, can be manufactured, for example, in powder form, in ceramic form, or in any other suitable form. The wavelength converting structure may be formed into one or more structures that are formed separately from and can be handled separately from the primary light source, such as a prefabricated glass or ceramic tile, or may be formed into a structure that is formed in situ with the light source, such as a conformal or other coating formed on or above the source.

In some embodiments, the wavelength converting structure may be powders that are dispersed for example in a transparent matrix, a glass matrix, a ceramic matrix, or any other suitable material or structure. SWIR phosphor dispersed in a matrix may be, for example, singulated or formed into a tile that is disposed over a light source. The glass matrix may be for example a low melting glass with a softening point below 1000° C., or any other suitable glass or other transparent material. The ceramic matrix material can be for example a fluoride salt such as CaF₂ or any other suitable material.

The SWIR phosphors, or combination of SWIR phosphors and additional IR phosphors, can be applied in powder from with e.g. particles in the 3-50 pm average diameter range, to form a wavelength converting structure. The powders may be dispersed in a curable polysiloxane type resin and applied by e.g. means of dispensing into packages comprising primary light emitting LEDs. The powders can also be mixed with a low melting glass powder and heated above the glass softening temperature to form phosphor in glass converter structures (PiG). Alternatively SWIR phosphors can be mixed into a silicone resin and casted or attached to a glass substrate to form a phosphor on glass structure (PoG).

Wavelength converting structure 108 may be formed, for example, by mixing the powder SWIR phosphor, or combination of powder SWIR phosphor and powder additional SWIR phosphor, with a transparent material such as silicone and dispensing or otherwise disposing it in a path of light. In powder form, the average particle size (for example, particle diameter) of the SWIR phosphors and additional IR phosphors may be at least 1 μm in some embodiments, no more than 50 μm in some embodiments, at least 5 μm in some embodiments, and no more than 20 μm in some embodiments. Individual SWIR phosphor particles, or powder SWIR phosphor layers, may be coated with one or more materials such as a silicate, a phosphate, and/or one or more oxides in some embodiments, for example to improve absorption and luminescence properties and/or to increase the material's functional lifetime.

Wavelength converting structures in which a second phosphor system and/or an additional second phosphor system is included, such as the wavelength converting structure 218 described with respect to FIG. 2, can be manufactured using the same methods described above.

The SWIR phosphor and the second phosphor, and/or the additional IR phosphor and additional second phosphor, may be mixed together in a single wavelength converting layer, or formed as separate wavelength converting layers. In a wavelength converting structure with separate wavelength converting layers, SWIR phosphor and the second phosphor, and/or the additional IR phosphor and additional second phosphor, may be stacked such that the second phosphor (and/or additional second phosphor) may be disposed between the SWIR phosphor (and/or the additional IR phosphor) and the light source, or the SWIR phosphor (and/or additional IR phosphor) may be disposed between the second phosphor (and/or additional second phosphor) and the light source.

FIGS. 4, 5, and 6 illustrate devices that combine an LED 1 and a wavelength converting structure 30. The wavelength converting structure 30 may be, for example, wavelength converting structure 108 including an SWIR phosphor as shown in FIG. 1, or wavelength converting structure 218 having an SWIR phosphor and a second phosphor as shown in FIG. 2, according to the embodiments and examples described above.

In FIG. 4, the wavelength converting structure 30 is directly connected to the LED 1. For example, the wavelength converting structure may be directly connected to the substrate 10 illustrated in FIG. 3, or to the semiconductor structure, if the substrate 10 is removed.

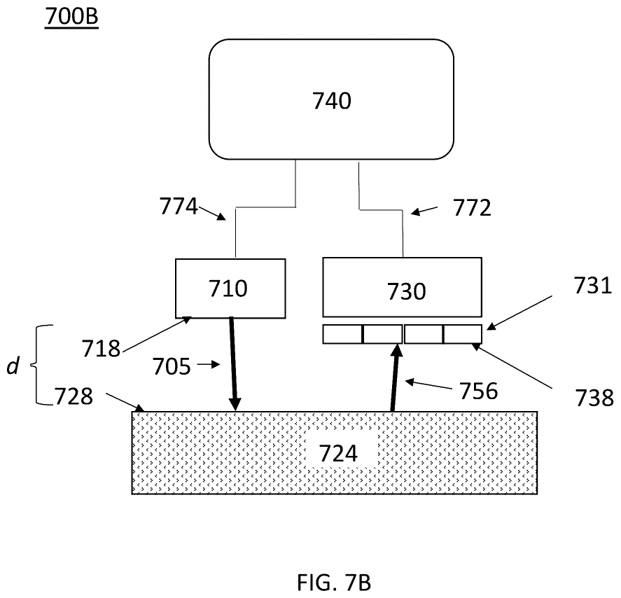

In FIG. 5, the wavelength converting structure 30 is disposed in close proximity to LED 1, but not directly connected to the LED 1. For example, the wavelength converting structure 30 may be separated from LED 1 by an adhesive layer 32, a small air gap, or any other suitable structure. The spacing between LED 1 and the wavelength converting structure 30 may be, for example, less than 500 μm in some embodiments.

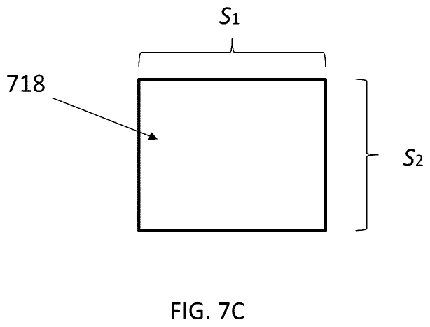

In FIG. 6, the wavelength converting structure 30 is spaced apart from LED 1.

The spacing between LED 1 and the wavelength converting structure 30 may be, for example, on the order of millimeters in some embodiments. Such a device may be referred to as a “remote phosphor” device.

The wavelength converting structure 30 may be square, rectangular, polygonal, hexagonal, circular, or any other suitable shape. The wavelength converting structure may be the same size as LED 1, larger than LED 1, or smaller than LED 1.

Multiple wavelength converting materials and multiple wavelength converting structures can be used in a single device.

A device may also include other wavelength converting materials in addition to the SWIR phosphor, second phosphor, additional IR phosphor, and/or additional second phosphor described above, such as, for example, conventional phosphors, organic phosphors, quantum dots, organic semiconductors, II-VI or III-V semiconductors, II-VI or III-V semiconductor quantum dots or nanocrystals, dyes, polymers, or other materials that luminesce.

Multiple wavelength converting materials may be mixed together or formed as separate structures.

In some embodiments, other materials may be added to the wavelength converting structure or the device, such as, for example, materials that improve optical performance, materials that encourage scattering, and/or materials that improve thermal performance. An example of such a material is (Al,Ga)₂O₃ as second phase in polycrystalline ceramics of the structurally disordered cubic garnet SWIR phosphors disclosed herein.

### IR Spectrometers Useful as Glucose Measurement Devices

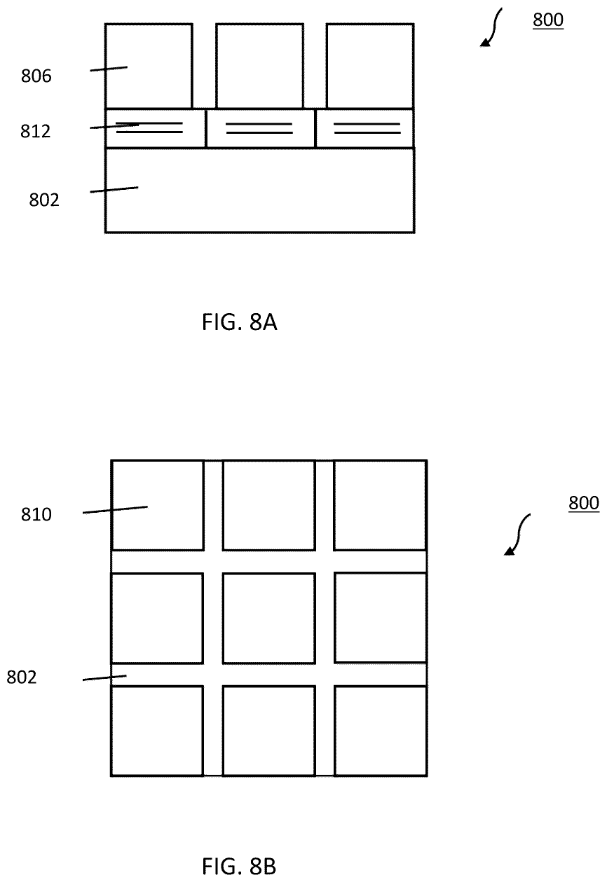

FIGS. 7A and 7B illustrate examples of IR spectrometers including SWIR light emitting devices with sensors (detectors) positioned for, respectively, absorption and reflectance spectroscopy. Light emitting devices, such as 101, 201 of FIGS. 1 and 2, having one or more of the SWIR phosphors disclosed herein may be used in spectrometer devices for IR absorption and/or reflection spectroscopy applications.

FIG. 7A shows a diagram of an IR spectrometer 700A with sensor/detector in a configuration useful for absorption spectroscopy. FIG. 7B shows a diagram of an IR spectrometer 700B with sensor/detector in a configuration useful for reflectance spectroscopy. In FIGS. 7A and 7B, the IR spectrometers 700A and 700B include an IR light emitting device 710 that may include one or more of the SWIR phosphors or combination of SWIR phosphor and additional IR phosphors (with or without second phosphor system, or additional second phosphor system, respectively), such as light emitting devices 101, 201 of FIG. 1 and FIG. 2, respectively. IR light source 710 may also be an IR light source array, such as array 800 described below with respect to FIGS. 8A and 8B.

IR spectrometers 700A and 700B further include a sensor/detector 730, which is an infrared light detector, for sensing the IR light. In particular, sensor/detector 730 is capable of sensing infrared light in the short wavelength range. Sensor/detector 730 may be, for example a photoresistor or photodiode that can be further combined with light guiding and/or diffracting elements 731. In one example, the sensor/detector 730 is specifically formed to detect IR light in a miniaturized device. For example, sensor/detector 730 may be a lead chalcogenide (PbS, PbSe) based photoresistor sensing elements, for example formed into a thin film PbS, which may detect IR radiation over the 1000-3000 nm wavelength range. To provide spectral resolution, the sensing elements such as PbS photoresistors may be combined with an array of optical filtering elements such as, for example, band pass filters. In another example, sensor/detector 730 may be and InGaAs type sensor.

The IR spectrometers 700A and 700B may further include, for example, a processor 740. Processor 740 may process data received from the sensor/detector 730. Processor 740 may also include a controller function, for controlling the IR light emitting device 710 and/or sensor/detector 730, and thus may include, for example, driving and readout electronics. In FIG. 7B, IR spectrometer 700B is shown having processor 740 separated from the IR light emitting device 710 and the sensor/detector 730, with the IR light emitting device connected to processor 740 through connector 774, and the sensor/detector 730 connected to processor 740 through connector 772. Connectors 774 and 772 may be, for example, physical wiring, or may be a wireless communication technology, for example Bluetooth®. Processor 740 can be connected directly as shown in FIG. 7A or remotely, as shown in FIG. 7B, with either of the IR lighting device 710 and sensor/detector 730 configurations shown in 700A or 700B.

In FIG. 7A, the IR spectrometer 700A is configured with the sensor/detector 730 positioned opposite the IR light emitting device 710, which is useful for absorption spectroscopy. IR spectrometer 700A may also include a place for a sample 720, if a sample is to be inserted into the IR spectrometer between the IR light emitting device 710 and the sensor detector 730, as shown in FIG. 7A.

In operation of IR spectrometer 700A, the IR light emitting device 710 emits IR light 705 from light emitting surface 718. Emitted IR light 705 may be a broad-band emission over a 1600-2200 nm range, or may be broader, over the 1100-2200 nm range, depending on the phosphor combinations in the wavelength converting structure of the IR light emitting device 710. The emitted light enters the sample 720, and IR light 706 of the IR absorption spectra exits the sample 720 to be detected by sensor/detector 730. The light guide and/or diffracting element 731 may filter certain wavelengths of the IR absorption spectra light 706 before reaching the sensor/detector 730. For example, the sample 720 may be a blood sample, and various analytes, for example glucose, may be detected, and the sensor/detector 730 may include light guide and/or diffracting element 731 which allows only certain of the wavelengths to pass through, and filters the remaining wavelengths. For instance, light guide and/or diffracting element may pass only those wavelengths useful for measurement of glucose.

In FIG. 7B, the IR spectrometer 700B is configured with the sensor/detector 730 positioned adjacent to the IR light emitting device 710. IR light emitting device 710 and sensor/detector 730 are both facing sample 724, which is useful for reflectance spectroscopy. Sample 724 may be, for example, skin tissue.

In operation on IR spectrometer 700B, the IR light emitting device 710 emits IR light 705 from light emitting surface 718. Emitted IR light 705 may be a broad-band emission over a 1600-2200 nm range, or may be broader, over the 1100-2200 nm range, depending on the phosphor combinations in the wavelength converting structure of the IR light emitting device 710. The emitted light 705 is incident on sample 724, and some portion of the IR light incident on sample 724 will be reflected back toward IR spectrometer 700B. The reflected IR light 756 is incident on a light receiving surface 738 of sensor/detector 730, which light receiving surface 738 may be on the light guide and/or diffraction element 731. The light guide and/or diffracting element 731 may filter certain wavelengths of the IR reflection spectra light 756 before reaching the sensor/detector 730. For example, sample 724 may be skin tissue, and the glucose levels may be detected using the reflected IR light 756 reflected off of the skin tissue.

IR spectrometers 700A and 700B may be glucose measurement devices for non-invasively monitoring patient glucose levels. In such glucose measurement devices, one or more IR light emitting devices 710 may be formed with structures and materials as described above under the heading “IR Light Emitting Devices having Wavelength Converting Structures Emitting with Temperature Stable Emissions at Glucose Absorption Maxima and Minima.” Such light sources can provide SWIR emissions having wavelength and intensity stable emission in the blood glucose absorption band wavelength ranges.

To detect glucose levels, the SWIR light in wavelength ranges around the wavelengths of the absorption maxima (1942 nm and 2098 nm), and minima (1890 nm and 2004 nm) of tissue glucose is detected in the reflectance light 756 reflected off of skin tissue sample 724. Thus, the glucose measurement devices has a light guide and/or diffracting element 731 that may be a segmented filter element that selectively passes at least two wavelength ranges reflected from skin tissue (where sample 724 is skin tissue). Thus, the segmented filter element may selectively pass wavelength in the spectral ranges of, for example, at least one of, 1942±6 nm and/or 2098 nm ±6 nm along with 1890±6 nm and/or 2004±6 nm, or for example 1942±0-12 nm and/or 2098 nm ±0-12 nm along with 1890±0-12 nm and/or 2004±0-12 nm. The light guide and/or diffracting element 731 may further include other segments that allow sensing of other wavelength ranges to allow sensing of other substances or to further improve sensing accuracy.

For IR reflectance spectrometer 700B, FIG. 7B shows the IR light emitting device 710 as separate from the detector/sensor 730, however, the two elements may be integrated into a single device and may be arranged directly in contact while adjacent. Different geometries may be used in the arrangement, so long as IR light 705 emitted from the IR light emitting device 710 is incident on the skin tissue sample 724, and reflected IR light 756 reaches the detector/sensor 730. In FIG. 7B, the IR spectrometer 700 is illustrated at a distance d from the surface 728 of the sample 724, which may be skin tissue. When incorporated in a device and in use, the light emitting surface 718 and light receiving surface 738 may be positioned directly onto or very near the surface of the skin 728, such that distance d is zero or relatively small. The distance d that works best may depend on the particular application and, for example, the position on the body in which the device is worn.

IR reflectance spectrometer 700B may be particularly useful as glucose measurement device when integrated into a smartphone, handheld device, or a wearable device, and in particular devices which are already worn by the user for other functions. For example, such IR reflectance spectrometers 700B may be integrated into wrist watches, or into hearing aids or ear buds inserted into the ear, or a dental type device for measurement within the mouth, for example, on the inner lip.

An advantage of using the SWIR light emitting devices in IR spectrometers as disclosed herein is that they allow true miniaturization of the device. FIG. 7C shows a plan view of the light emitting surface 718 of an IR light emitting device 710. The surface area s1 X s2 of light emitting surface 718 may have any useful size, and may be, for example, less than 1 cm×1 cm, for instance as small as 1 mm×1 mm, or as small as 0.5 mm×0.5 mm. The IR lighting emitting surface of FIG. 7C is illustrated as monolithic, but as light emitting device 710 may be, for example, an array 800, the light emitting surface may be composed of an array of densely packed emitters, either as formed from combining separate individual pcLEDs, or as formed monolithically on a shared substrate. The other dimensions of the IR light emitting device 710 may remain less than the cm range, for instance within the mm dimensions. Similarly, sensor/detector 730 and processor 740 (if connected directly) may be less than 1 cm in dimensions, for instance in the mm range. Thus, the IR reflectance spectrometer 700B may have a size on the order of a cm or less, which makes incorporation into wearable devices feasible.

### IR Emitting Arrays

FIGS. 8A-8B show, respectively, cross-sectional and top views of an array 800 of SWIR pcLEDs 810, which SWIR pcLEDs 810 may be structured as lighting device 101, 201, or 710, as shown in FIGS. 1, 2 and 7, respectively, that include a wavelength converter including one or more of the SWIR phosphors as disclosed herein included in phosphor pixels 806 with semiconductor diode 812 disposed on a substrate 802. The wavelength converters may include one or more SWIR phosphors or combination of SWIR phosphors and additional IR phosphors, with or without second phosphor systems and/or additional second phosphor systems as described above. Such an array may include any suitable number of SWIR pcLEDs arranged in any suitable manner. In the illustrated example, the array 800 is depicted as formed monolithically on a shared substrate, but alternatively an array of SWIR pcLEDs may be formed from separate individual pcLEDs. Substrate 802 may optionally comprise CMOS circuitry for driving the LED and may be formed from any suitable materials.

Although FIGS. 8A-8B, show a three-by-three array of nine pcLEDs, such arrays may include for example tens, hundreds, or thousands of LEDs. Individual LEDs (pixels) may have widths (e.g., side lengths) in the plane of the array, for example, less than or equal to 1 millimeter (mm), less than or equal to 500 microns, less than or equal to 100 microns, or less than or equal to 50 microns. LEDs in such an array may be spaced apart from each other by streets or lanes having a width in the plane of the array of, for example, hundreds of microns, less than or equal to 100 microns, less than or equal to 50 microns, less than or equal to 10 microns, or less than or equal to 5 microns. Although the illustrated examples show rectangular pixels arranged in a symmetric matrix, the pixels and the array may have any suitable shape or arrangement.

LEDs having dimensions in the plane of the array (e.g., side lengths) of less than or equal to about 50 microns are typically referred to as microLEDs, and an array of such microLEDs may be referred to as a microLED array.

An array of LEDs, or portions of such an array, may be formed as a segmented monolithic structure in which individual LED pixels are electrically isolated from each other by trenches and/or insulating material, but the electrically isolated segments remain physically connected to each other by portions of the semiconductor structure.

The individual LEDs in an LED array may be individually addressable, may be addressable as part of a group or subset of the pixels in the array, or may not be addressable. Thus, light emitting pixel arrays are useful for any application requiring or benefiting from fine-grained intensity, spatial, and temporal control of light distribution. These applications may include, but are not limited to, precise special patterning of emitted light from pixel blocks or individual pixels. Depending on the application, emitted light may be spectrally distinct, adaptive over time, and/or environmentally responsive. Such light emitting pixel arrays may provide pre-programmed light distribution in various intensity, spatial, or temporal patterns. The emitted light may be based at least in part on received sensor data and may be used for optical wireless communications. Associated electronics and optics may be distinct at a pixel, pixel block, or device level.

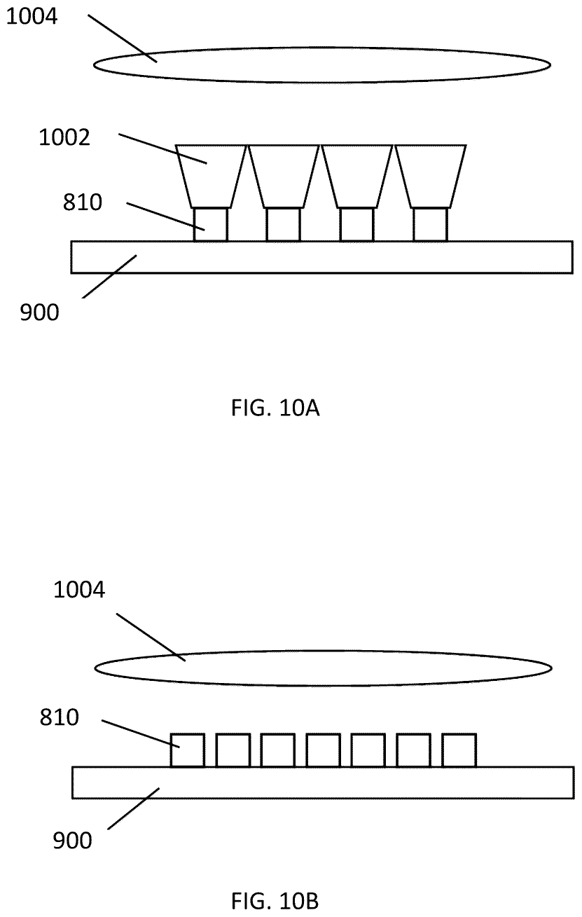

As shown in FIGS. 9A-9B, an SWIR pcLED array 800 may be mounted on an electronics board 900 comprising a power and control module 902, a sensor module 904, and an LED attach region 906. Power and control module 902 may receive power and control signals from external sources and signals from sensor module 904, based on which power and control module 902 controls operation of the LEDs. Sensor module 904 may receive signals from any suitable sensors, for example from temperature or light sensors. Alternatively, SWIR pcLED array 800 may be mounted on a separate board (not shown) from the power and control module and the sensor module.

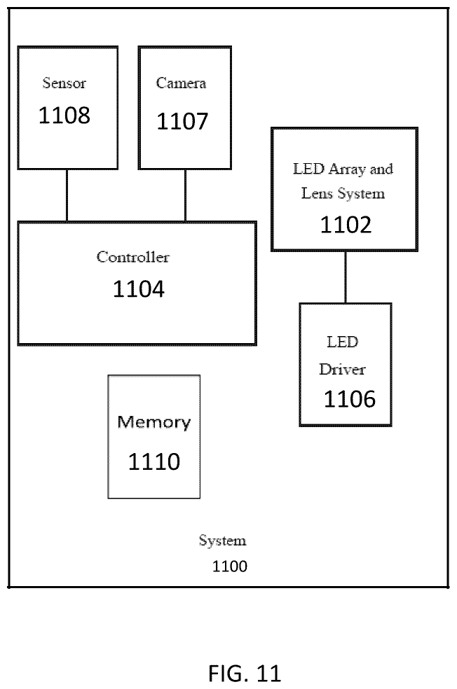

Individual SWIR pcLEDs may optionally incorporate or be arranged in combination with a lens or other optical element located adjacent to or disposed on the phosphor layer. Such an optical element, not shown in the figures, may be referred to as a “primary optical element”. In addition, as shown in FIGS. 10A-lOB an SWIR pcLED array 800 (for example, mounted on an electronics board 900) may be arranged in combination with secondary optical elements such as waveguides, lenses, or both for use in an intended application. In FIG. 10A, light emitted by SWIR pcLEDs 810 is collected by waveguides 1002 and directed to projection lens 1004. Projection lens 1004 may be a Fresnel lens, for example. In Figure lOB, light emitted by SWIR pcLEDs 810 is collected directly by projection lens 1004 without use of intervening waveguides. This arrangement may be particularly suitable when SWIR pcLEDs can be spaced sufficiently close to each other and may also be used in various applications. A microLED display application may use similar optical arrangements to those depicted in FIGS. 10A-lOB, for example. Generally, any suitable arrangement of optical elements may be used in combination with the LED arrays described herein, depending on the desired application.

An array of independently operable LEDs may be used in combination with a lens, lens system, or other optical system (e.g., as described above) to provide illumination that is adaptable for a particular purpose. For example, in operation such an adaptive lighting system may provide illumination that varies by wavelength and/or intensity across an illuminated sample or object and/or is aimed in a desired direction. A controller can be configured to receive data indicating locations and spectral characteristics of objects or persons in a sample, and based on that information control LEDs in an LED array to provide illumination adapted to the scene. Such data can be provided for example by an image sensor, or optical (e.g. laser scanning) or non-optical (e.g. millimeter radar) sensors. Such adaptive illumination is increasingly important for mobile devices, VR, and AR applications.

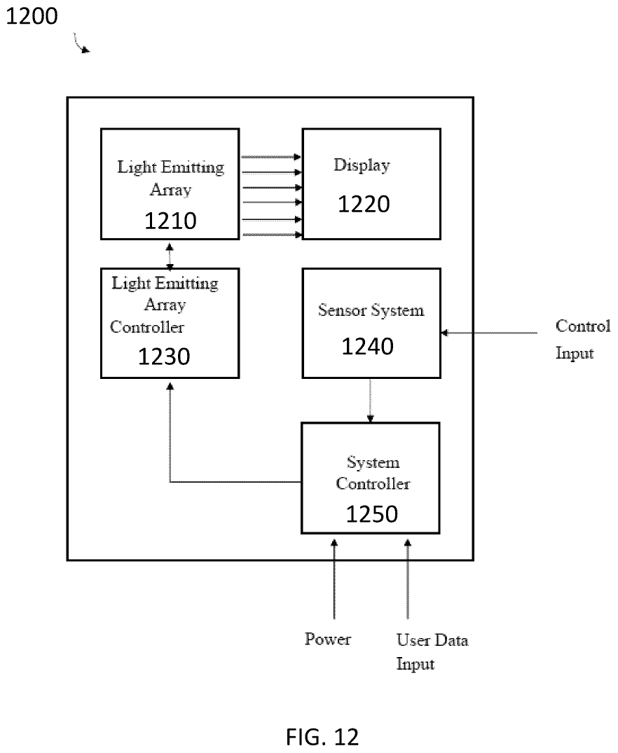

FIG. 11 schematically illustrates an example camera flash system 1100 comprising an SWIR pcLED array and lens system 1102, which may be similar or identical to the systems described above. Flash system 1100 also may include an SWIR pcLED driver 1106 that is controlled by a controller 1104, such as a microprocessor. Controller 1104 may also be coupled to a camera 1107 and to sensors 1108, and operate in accordance with instructions and profiles stored in memory 1110. Camera 1107 and adaptive illumination system 1102 may be controlled by controller 1104 to match their fields of view.

Sensors 1108 may include, for example, positional sensors (e.g., a gyroscope and/or accelerometer) and/or other sensors that may be used to determine the position, speed, and orientation of system 1100. The signals from the sensors 1108 may be supplied to the controller 1104 to be used to determine the appropriate course of action of the controller 1104 (e.g., which LEDs are currently illuminating a target and which LEDs will be illuminating the target a predetermined amount of time later).

In operation, illumination from some or all pixels of the LED array in 1102 may be adjusted - deactivated, operated at full intensity, or operated at an intermediate intensity. Beam focus or steering of light emitted by the LED array in 1102 can be performed electronically by activating one or more subsets of the pixels, to permit dynamic adjustment of the beam shape without moving optics or changing the focus of the lens in the lighting apparatus.

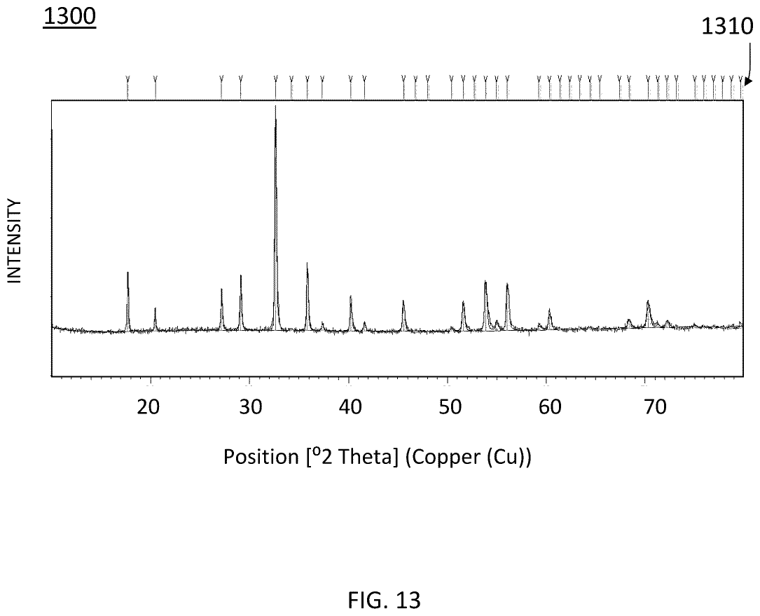

FIG. 12 schematically illustrates an example display (e.g., AR/VR/MR) system 1200 that includes an adaptive light emitting array 1210, display 1220, a light emitting array controller 1230, sensor system 1240, and system controller 1250. Control input is provided to the sensor system 1240, while power and user data input is provided to the system controller 1250. In some embodiments modules included in system 1200 can be compactly arranged in a single structure, or one or more elements can be separately mounted and connected via wireless or wired communication. For example, the light emitting array 1210, display 1220, and sensor system 1240 can be mounted on a headset or glasses, with the light emitting controller and/or system controller 1250 separately mounted.

The light emitting array 1210 may include one or more adaptive light emitting arrays, as described above, for example, that can be used to project light in graphical or object patterns that can support AR/VR/MR systems. In some embodiments, arrays of microLEDs can be used.

System 1200 can incorporate a wide range of optics in adaptive light emitting array 1210 and/or display 1220, for example to couple light emitted by adaptive light emitting array 1210 into display 1220.

Sensor system 1240 can include, for example, external sensors such as cameras, depth sensors, or audio sensors that monitor the environment, and internal sensors such as accelerometers or two or three axis gyroscopes that monitor an AR/VR/MR headset position. Other sensors can include but are not limited to air pressure, stress sensors, temperature sensors, or any other suitable sensors needed for local or remote environmental monitoring. In some embodiments, control input can include detected touch or taps, gestural input, or control based on headset or display position.

In response to data from sensor system 1240, system controller 1250 can send images or instructions to the light emitting array controller 1230. Changes or modification to the images or instructions can also be made by user data input, or automated data input as needed. User data input can include but is not limited to that provided by audio instructions, haptic feedback, eye or pupil positioning, or connected keyboard, mouse, or game controller.

### EXAMPLES

In the following examples, compositions of SWIR phosphors disclosed herein and pcLEDs including these SWIR phosphors are described.

### Example 1

Example 1 describes the synthesis of SWIR phosphor compositions of Gd_{2.367}Ho_{0.01}Tm_{0.152}Sc_{1.6}Lu_{0.27}Ga_{1.8}Al_{1.78}Cr_{0.04}O₁₂. SWIR phosphor of composition Gd_{2.367}Ho_{0.01}Tm_{0.152}Sc_{1.6}Lu_{0.27}Ga_{1.8}Al_{1.78}Cr_{0.04}O₁₂ were synthesized by combining 28.7 g gadolinium oxide (Treibacher, >99.98%), 7.66 g scandium oxide (Treibacher, 99.99%), 3.65 g luthetium oxide (Rhodia, 99.99%), 11.6 g gallium oxide (Dowa Electronics Materials, 4N) 0.236 g chromium(II) oxide (Alfa Aesar, 98%), 6.22 g aluminum oxide (Baikowski, SP-DBM), 0.128 g holmium oxide (K. Rasmus & Co, 4N), 2.027 g thulium oxide (Alfa Aesar, >99.9%) and 1.01 g gadolinium fluoride (Materion, 4N). These compounds were mixed by planetary ball milling. The mixture was then fired in an air atmosphere at 1540° C. for 8 hours, followed by ball milling, and next fired in an air atmosphere at 1510° C. for 8 hours. After the second firing of the mixture, crushing and ball milling of the mixture is performed to obtain a powder of the SWIR phosphor. The SWIR phosphor powder was washed with water, dried at 300° C. in air and finally screened through a 50 pm sieve.

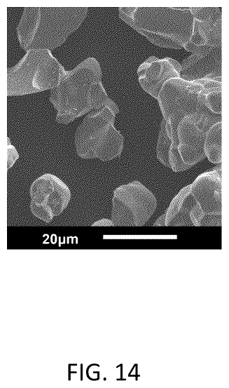

FIG. 13 shows the X-ray powder pattern 1300 (copper radiation) of the Gd_{2.367}Ho_{0.01}Tm_{0.152}Sc_{1.6}Lu_{0.27}Ga_{1.8}Al_{1.78}Cr_{0.04}O₁₂ SWIR phosphor obtained in Example 1. The grey lines 1310 represent the position and heights of fitted reflections calculated with the cubic garnet structure model. Example 1 shows a cubic lattice constant of 12.266 A and a calculated density of 6.38 g/cm³.

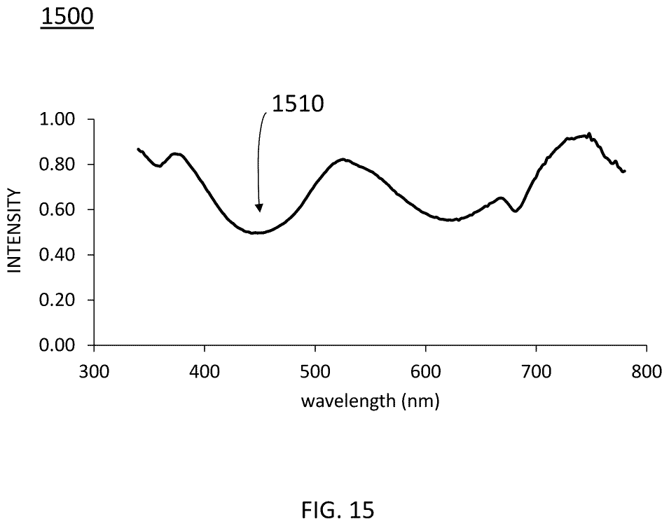

FIG. 14 shows a scanning electron microscopy (SEM) image 1400 of the Gd_{2.367}Ho_{0.01}Tm_{0.152}Sc_{1.6}Lu_{0.27}Ga_{1.8}Al_{1.78}Cr_{0.04}O₁₂ SWIR phosphor powder obtained in Example 1.

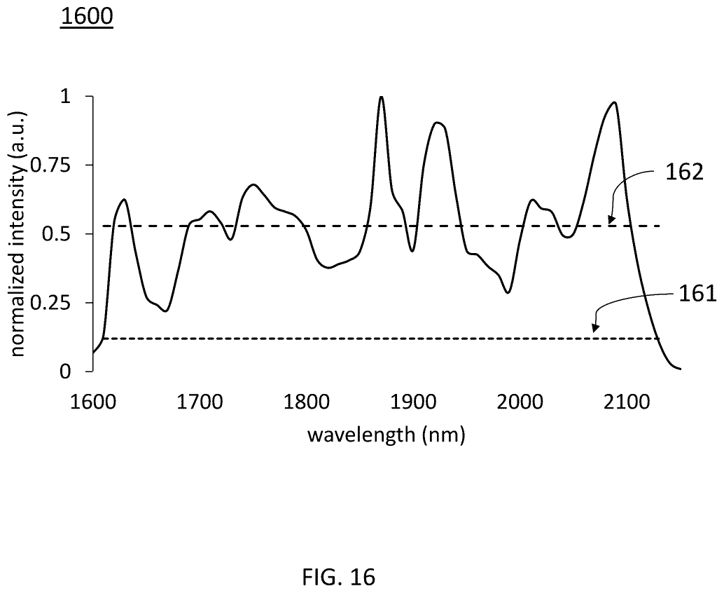

FIG. 15 shows a power reflectance spectrum 1500 of Example 1 in the visible spectral range. The reflection minimum 1510 in the visible spectral range is in the blue spectral region at around 450 nm.

### Example 2

Example 2 describes the formation of an SWIR pcLED that includes the SWIR phosphor synthesized in Example 1. An SWIR pcLED including the SWIR phosphor of Example 1 was formed by mixing a powder of the Gd_{2.367}Ho_{0.01}Tm_{0.152}Sc_{1.6}Lu_{0.27}Ga_{1.8}Al_{1.78}Cr₀ synthesized in Example 1 with a thermally curable silicone resin (phosphor/silicone weight ratio 1.6) under vacuum. The mixture of SWIR phosphor and thermally curable silicone resin was dispensed into a midpower LED packages containing InGaN blue emitters (emission wavelength ˜450 nm).

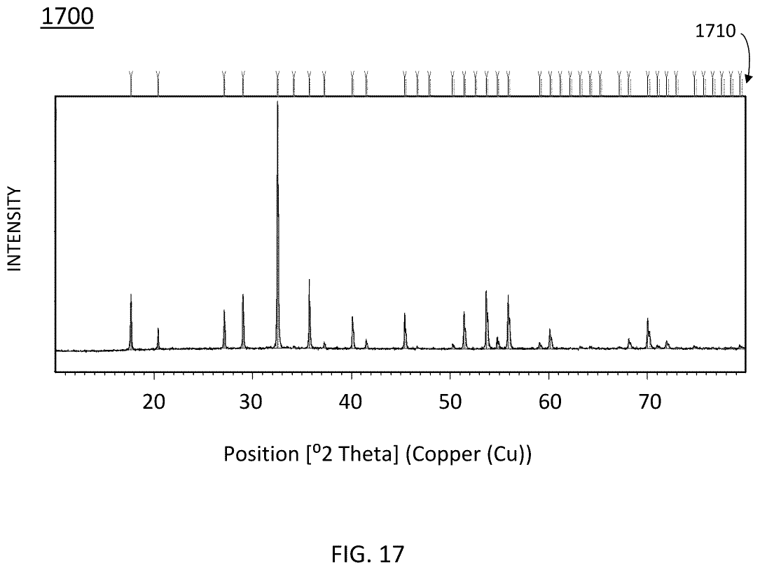

FIG. 16 shows the normalized short-wave infrared emission spectrum 1600 of the SWIR pcLED formed in Example 2. The emission spectrum shows that emission from the SWIR pcLED covers the range from 1610-2130 nm. For the wavelength range 1610-2130 nm the minimum 161 and average 162 emission power relative to the maximum emission power is larger than 12% and 53% (dotted and dashed lines), respectively.

### Example 3

Example 3 describes the synthesis of SWIR phosphor compositions of Gd_{2.59}Tm_{0.24}Ho_{0.02}Sc_{0.75}Lu_{0.3}Ga₂Al₂Cr_{0.1}O₁₂. SWIR phosphor of composition Gd_{2.59}Tm_{0.24}Ho_{0.02}Sc_{0.75}Lu_{0.3}Ga₂Al₂Cr_{0.1}O₁₂ were synthesized by combining 29.56 g gadolinium oxide (Treibacher, >99.98%), 3.39g scandium oxide (Treibacher, 99.99%), 3.87 g luthetium oxide (Rhodia, 99.99%), 12.34 g gallium oxide (Dowa Electronics Materials, 4N) 0.493 g chromium(II) oxide (Alfa Aesar, 98%), 6.22 g aluminum oxide (Baikowski, SP-DBM), 0.255 g holmium oxide (K. Rasmus & Co, 4N), 2.995 g thulium oxide (Alfa Aesar, >99.9%) and 1.04 g gadolinium fluoride (Materion, 4N). The compounds were mixed by planetary ball milling. The mixture was then fired in an air atmosphere at 1540° C. for 8 hours, followed by ball milling, and next fired in an air atmosphere at 1510° C. for 8 hours. After the second firing of the mixture, crushing and ball milling of the mixture is performed to obtain a powder of the SWIR phosphor. The SWIR phosphor powder was washed with water, dried at 300° C. in air and finally screened through a 50 pm sieve.

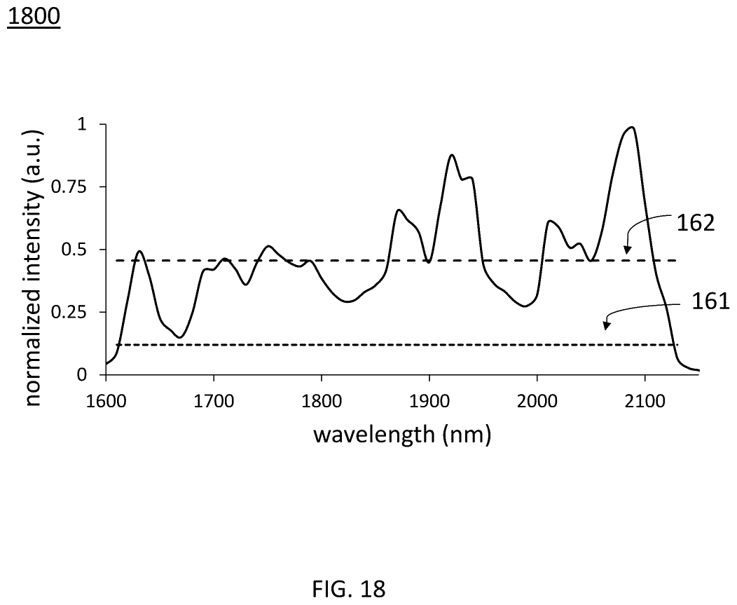

FIG. 17 shows the X-ray powder pattern 1700 (copper radiation) of SWIR phosphor compositions of Gd_{2.59}Tm_{0.24}Ho_{0.02}Sc_{0.75}Lu_{0.3}Ga₂Al₂Cr_{0.1}O₁₂ formed in Example 3. The grey lines 1710 represent the position and heights of fitted reflections calculated with the cubic garnet structure model. Example 3 shows a cubic lattice constant of 12.301 A and a calculated density of 6.62 g/cm³.

### Example 4

Example 4 describes the formation of an SWIR pcLED that includes the SWIR phosphor synthesized in Example 3. An SWIR pcLED including the SWIR phosphor of Example 3 was formed by mixing a powder of the Gd_{2.59}Tm_{0.24}Ho_{0.02}Sc_{0.75}Lu_{0.3}Ga₂Al₂Cr_{0.1}O₁₂ synthesized in Example 3 with a thermally curable silicone resin (phosphor/silicone weight ratio 1.6) under vacuum. The mixture of SWIR phosphor and thermally curable silicone resin was dispensed into a midpower LED packages containing InGaN blue emitters (emission wavelength ˜450 nm).

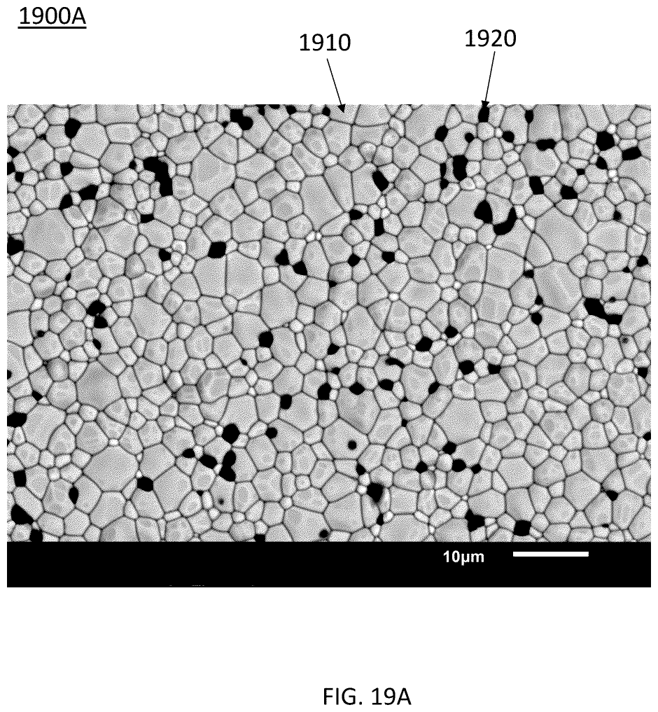

FIG. 18 shows the normalized short-wave infrared emission spectrum 1800 of the SWIR pcLED formed in Example 4. The emission spectrum shows that emission from the SWIR pcLED covers the 1600-2130 nm spectral range. For the wavelength range 1610-2130 nm the minimum 181 and average 182 emission power relative to the maximum emission power is larger than 12% and 46% (dotted and dashed lines), respectively.

### Examples 5a and 5b

Examples 5a and 5b describe the formation of wavelength converting structures that are a composite ceramic plate including the SWIR garnet phosphor composition Gd₂Ho_{0.013}Tm_{0.2}Sc_{0.67}Lu_{0.24}Ga_{1.6}Al_{3.2}Cr_{0.08}O₁₂ as the main polycrystalline phase and an additional (Al,Ga)₂O₃ as the minority phase. Examples 5b differ from example 5a slightly in the source of materials used, and final thickness of the phosphor layer of phosphor composition. Example 5b also includes a dichroic coating layer to form a band pass filter useful for use in light emitting devices that are used, for example, in glucose sensing devices.

### Example 5a: The SWIR phosphor composition

Gd₂Ho_{0.013}Tm_{0.2}Sc_{0.67}Lu_{0.24}Ga_{1.6}Al_{3.2}Cr_{0.08}O₁₂ was prepared by combining 89.92 g gadolinium oxide (Treibacher, >99.98%), 11.58 g scandium oxide (Treibacher, 99.99%), 11.85 g luthetium oxide (Rhodia, 99.99%), 37.18 g gallium oxide (Dowa Electronics Materials, 4N), 1.512 g chromium(II) oxide (Alfa Aesar, 98%), 40.46 g aluminum oxide (Baikowski, SP-DBM), 0.611 g holmium oxide (K. Rasmus & Co, 4N), and 9.57 g thulium oxide (Alfa Aesar, >99.9%). These compounds were mixed in 99 g ethanol and 107 pl tetraethylorthosilicate (Merck, p.a.) by means of ball milling with a dispersant added (2 wt % Malialim AKM-0531) until an average particle size of 0.72 pm was reached. After addition of a polyvinylbutyral binder and plasticizer system (Sekisui BL-5, G-260), ceramic tapes were casted, dried, stacked and laminated. After de-bindering at 600° C., the ceramic plates were sintered at 1580° C. for 8 hrs in air atmosphere. The obtained composite ceramics with a thickness of 197 pm mainly crystallize in the cubic garnet structure with a lattice constant a₀=12.160 A with some (Al,Ga)₂O₃ secondary phase.

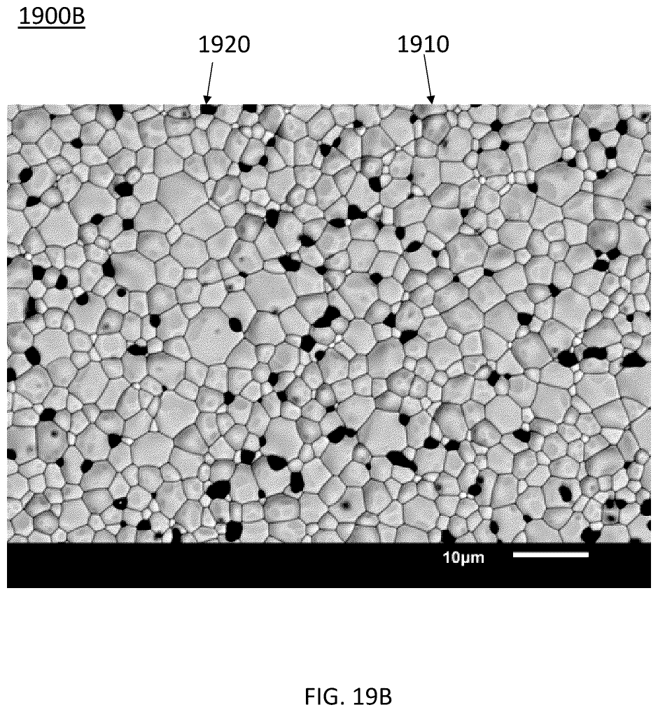

FIG. 19A shows a scanning electron micrograph 1900A of the as sintered SWIR phosphor ceramic. Light ceramic grains 1910 are the garnet phosphor phase while the dark ceramic grains 1920 are made up from the (Al,Ga)₂O₃ secondary phase.

Example 5b: The SWIR ceramic composite converter of composition of Example 5a (Gd₂O₃- 0.0065 Ho₂O₃-0.1 Tm₂O₃-0.33 Sc₂O₃-0.12 Lu₂O₃-0.8 Ga₂O₃- 0.04 Cr₂O₃- 1.6 Al₂O₃) can also be formed into a thinner ceramic, which may be useful for miniaturizing devices containing the SWIR light emitting devices disclosed herein. The SWIR ceramic was prepared using 89.92 g gadolinium oxide (Treibacher, 3N8), 11.58 g scandium oxide (Treibacher, 4N), 11.85 g lutetium oxide (Rhodia, 4N), 37.18 g gallium oxide (Dowa, 4N), 1.512 g chromium(III) oxide (Alfa Aesar, 2N), 40.46 g alumina (Baikowski, SP-DBM), 0.611 g holmium oxide (Treibacher, 4N) and 9.570 g thulium oxide (Treibacher, 4N) mixed in ethanol with a dispersant system (Malialim, tetraethoxysilane, Merck p.a.) by ball milling. The mixture was casted into thin ceramic tapes after addition of a poly vinyl butyral binder and plasticizer vehicle (Sekisui). After drying the tapes were stacked and laminated into ceramic green bodies, which are sintered ceramic bodies that have some mechanical strength due to the binder material as are understood by persons having ordinary skill in the art. that were de-bindered and sintered in air atmosphere at 1580° C. to obtain flat ceramic tiles with a thickness of 140 pm, which is 57 pm thinner than the ceramics of Example 5a.

The sintered composite ceramics of Example 5b mainly show microcrystalline grains crystallized in the cubic garnet structure type with a lattice constant of 12.160 A. FIG. 19B shows a scanning electron micrograph 1900B of the sintered SWIR phosphor ceramic. As in FIG. 19A, light ceramic grains 1910 are the garnet phosphor phase while the dark ceramic grains 1920 are made up from the (Al,Ga)₂O₃ secondary phase.

### Example 6a

The SWIR phosphor composite ceramics of Example 5a were coated with silica and niobia oxide layers according to the recipe in the following Table 1 to obtain a dichroic coating. The coating was applied on the surfaces of the as-sintered ceramics by reactive sputtering with silicon and niobium metal targets and oxygen as the reactive gas.

TABLE 1

| Layer # | Oxide | Thickness (nm) |
|---|---|---|
| 1 | SiO2 | 92.72 |
| 2 | Nb2O5 | 52.66 |
| 3 | SiO2 | 77.23 |
| 4 | Nb2O5 | 39.97 |
| 5 | SiO2 | 83.03 |
| 6 | Nb2O5 | 51.59 |
| 7 | SiO2 | 88.73 |
| 8 | Nb2O5 | 49.94 |
| 9 | SiO2 | 117.10 |
| 10 | Nb2O5 | 68.72 |
| 11 | SiO2 | 123.47 |
| 12 | Nb2O5 | 82.25 |
| 13 | SiO2 | 133.99 |
| 14 | Nb2O5 | 85.52 |
| 15 | SiO2 | 155.73 |
| 16 | Nb2O5 | 106.80 |
| 17 | SiO2 | 140.12 |
| 18 | Nb2O5 | 84.95 |

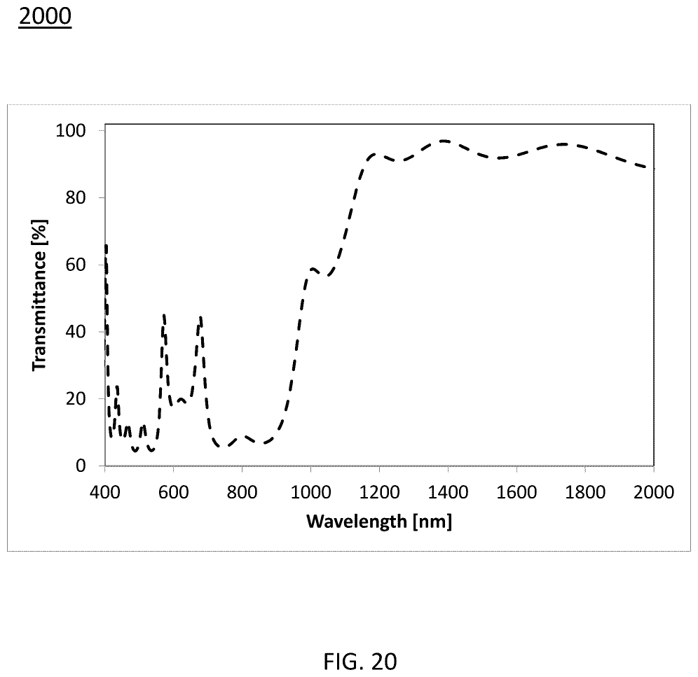

FIG. 20 shows a graph 2000 of the light transmission as a function of wavelength for the coated ceramic of this example obtained for an incidence angle of 0°.

### Example 6b

The thin SWIR phosphor ceramic tiles formed in Example 5b were coated with a dichroic coating layer (DCF) as described above in Example 6b. The DCF was formed of 9 layer pairs of alternating silica and niobia layers which were deposited onto the top surface of the Example 5b ceramic top surface by reactive sputter coating to form an optical long pass filter element. The DCF had a total thickness of 1.63 pm was formed on top of the flat ceramic tiles.

### Example 7

After dicing the ceramics made in Example 6a into platelets of size 1060×1060 μm, the obtained converter structures were attached (with the non-coated surfaces) to 440 nm emitting InGaN primary LED (LUXEON™, Lumileds) light sources having 1 mm² light emitting surfaces. The converter structures were attached with the non-coated surfaces disposed on the LED.

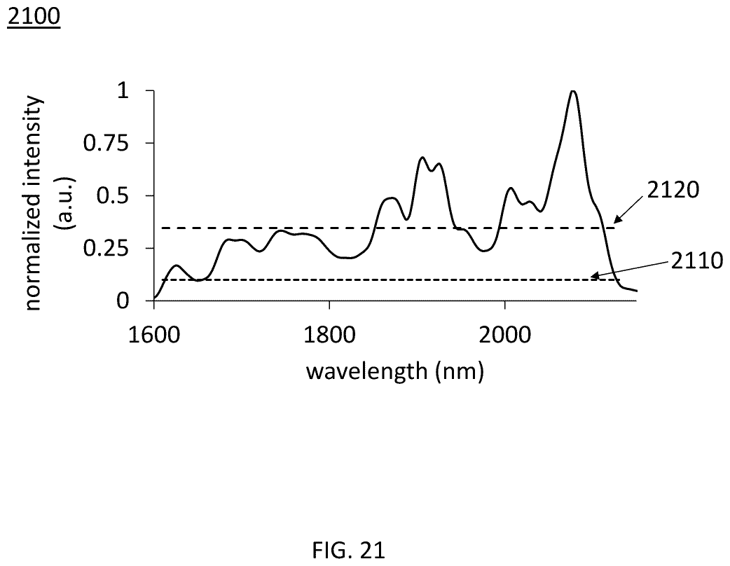

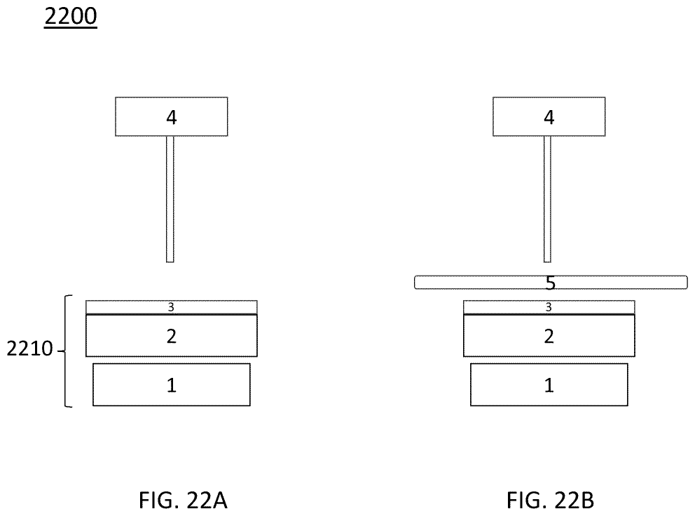

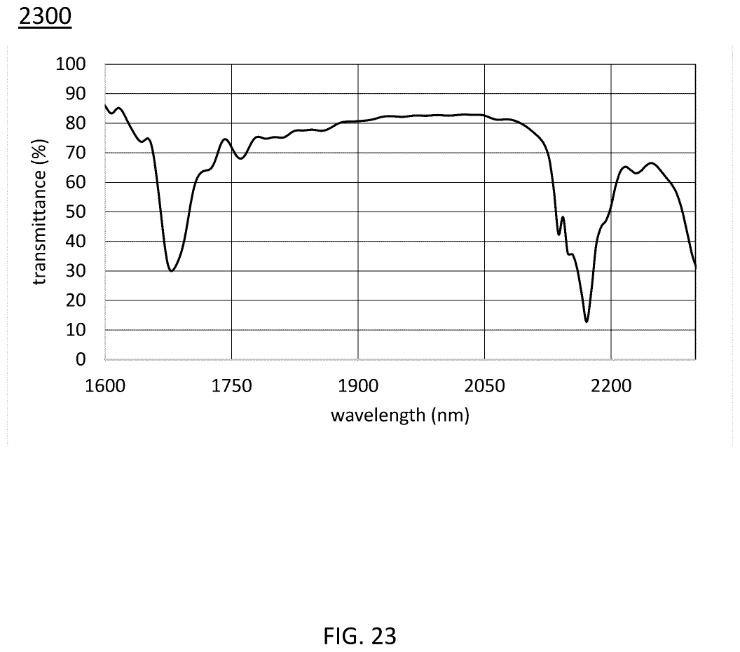

FIG. 21 shows the SWIR emission spectrum 2100 of the phosphor converted LEDs formed in this example. As can be seen in FIG. 21, the emission spectrum shows that emission from the SWIR pcLED covers the 1600-2130 nm spectral range. For the wavelength range 1610-2130 nm the minimum 2110 and average 2120 emission power relative to the maximum emission power is larger than 10% and 35%, respectively. The apparatus shown in FIGS. 22A and 22B was used to test the SWIR pcLED of Example 7 as a light source for spectroscopy. In FIGS. 22A and 22B, test apparatus 2200 includes the SWIR pcLED formed in this example 2210, including the blue light emitting InGaN primary LED 1 with the wavelength converting structure 2 with the dichroic coating 3 formed in Example 6a attached. The SWIR pcLED 2210 is positioned close to an IR spectrometer fiber optic sensor 4. FIG. 22B shows a test sample (polystyrene) 5., The SWIR pcLED 2210 was brought into proximity (10-20 mm distance) of the fiber optic of a Nanoquest® FT-IR spectrometer 4 (Ocean Insight B.V.), and first a reference spectrum was recorded, before a polystyrene test sample 5 was placed in the light path (FIG. 22). FIG. 23 shows the FT-IR spectrum of the polystyrene test sample calculated from the reference spectrum and the spectrum with the test sample in the light path.

### Example 8

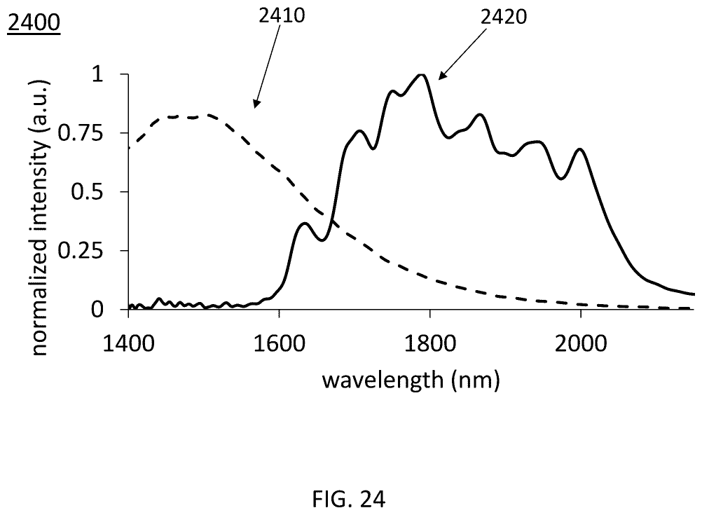

Example 8 describes the formation of a light source for spectroscopy that includes two different phosphors formed in two different wavelength converting structures to extend the light source emission to shorter wavelengths. The light source of this example includes two wavelength converting structures: (1) a first wavelength converting structure including the SWIR phosphor Gd_{2.32}Tm_{0.18}Sc_{1.5}Lu_{0.3}Ga_{1.81}Al_{1.81}Cr_{0.1}O₁₂ disclosed herein, and (2) a second ceramic wavelength converting structure including a garnet structure according to the specifications given U.S. patent application Ser. No. 17/035,233, filed Sep. 23, 2020, titled “SWIR pcLED and Phosphor Emitting in the 1100-1700 nm Range” with a composition Gd₃Ga_{3.7}ScAl_{0.18}Ni_{0.02}Zr_{0.021}Cr_{0.1}O₁₂ and a lattice constant a₀=12.3222 Å.. The first wavelength converting structure was formed as a ceramic plate including the SWIR garnet phosphor composition Gd_{2.32}Tm_{0.18}Sc_{1.5}Lu_{0.3}Ga_{1.81}Al_{1.81}Cr_{0.1}O₁₂ and was manufactured using the process as described for example 5. For example 8, 78.251 g gadolinium oxide (Treibacher, 3N5), 19.451 g scandium oxide (Treibacher, 4N), 11.114 g luthetium oxide (Rhodia, 4N), 31.54 g gallium oxide (Dowa Elecronics Materials, 4N), 1.41 g chromium(III) oxide (Alfa Aesar, 99%), 17.17 g aluminum oxide (Baikowski, SP-DBM), 6.445 g thulium oxide (Treibacher, 4N) and 110 pl tetraethylorthosilicate (Merck, p.a.) were milled in ethanol until an average particle size of 0.87 pm was reached. After the forming and firing steps as described for Example 5 ceramic SWIR phosphor ceramics with a unit cell constant a₀=12.293 A are being obtained. The second wavelength converting structure was formed as described in U.S. patent application Ser. No. 17/035,233, filed Sep. 23, 2020, titled “SWIR pcLED and Phosphor Emitting in the 1100-1700 nm Range. The first and second ceramic converter structures including, respectively, the Gd₃Ga_{3.7}ScAl_{0.18}Ni_{0.02}Zr_{0.02}ICr_{0.1}O₁₂ and Gd_{2.32}Tm_{0.18}Sc_{1.5}Lu_{0.3}Ga_{1.81}Al_{1.81}Cr_{0.1}O₁₂ phosphors were mounted on 440 nm emitting LED primary light sources to obtain an illumination system with the spectral power distribution in the SWIR wavelength range 2400 shown in FIG. 24. In FIG. 24, the dashed line 2410 shows the spectral power distribution of the illumination system with only the Gd₃Ga_{3.7}ScAl_{0.18}Ni_{0.02}Zr_{0.021}Cr_{0.1}O₁₂ phosphor material excited by the blue emitting primary LED light source, while the dotted line 2420 shows the spectral power distribution of the illumination system with only the Gd_{2.32}Tm_{0.18}Sc_{1.5}Lu_{0.3}Ga_{1.81}Al_{1.81}Cr_{0.1}O₁₂ phosphor material excited by the blue emitting primary LED light source.

### Example 9

Finished ceramic tiles from Example 6b having the DCF coating were diced into smaller pieces (1 mm×1 mm) and attached with the surface not having the DCF coating onto a 1 mm² blue emitting InGaN LED emitter. After applying a side coating consisting of titania particle loaded silicone resin onto the InGaN LED and ceramic converter side walls, a SWIR emitting pcLED was obtained that can be used for tissue glucose sensing applications.

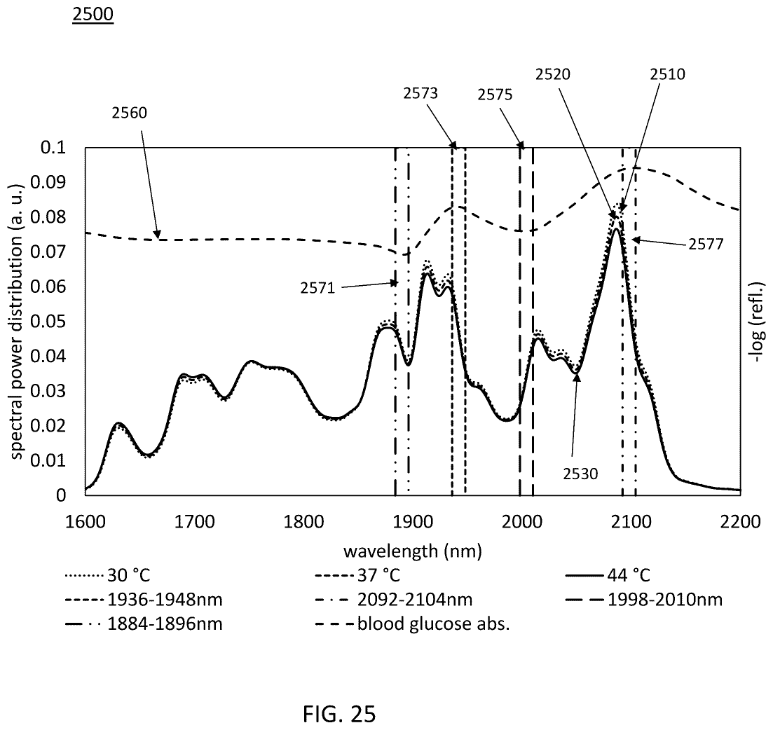

FIG. 25 shows the spectral power distribution 2500 measured in DC mode for different ambient temperatures for the SWIR light emitting device formed in this Example 9. The emission spectrum of the SWIR light emitting device, at 150 mA DC drive current, was measured between 1600 nm and 2200 nm at three different temperatures: 30° C. (2510), 37° C. (2520), and 44° C. (2530). As can be seen in FIG. 25, the spectral power emissions at the three different temperatures essentially overlap. The dashed spectra 2560 shows the absorption spectra for glucose between 1600 nm and 2200 nm superimposed on the measured emissions spectra for reference. The four wavelength ranges (in 12 nm intervals) at which tissue glucose has an maxima or a minima are indicated with the vertical dashed lines as follows: 1884-1896 nm (2571), 1936-1948 nm (2573), 1998-2010 nm (2575), and 2092-2104 nm (2577). As can be seen, within those four useful wavelength ranges there is essentially no change in the spectral power distribution at the three different temperatures. Thus, the spectral power distribution is very stable for the claimed wavelength ranges under varying ambient temperature.

### Example 10

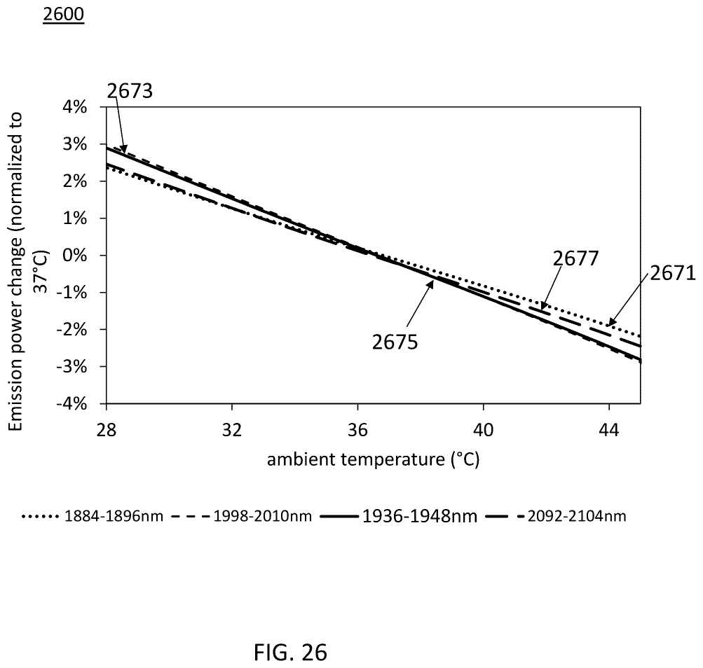

An IR spectrometer, such as the IR spectrometer 700B shown in FIG. 7B was formed using the SWIR light emitting device of Example 9 as the IR light emitting device 710. The SWIR light emitting device of Example 9 was combined with a SWIR sensor element (sensor/detector 730 of FIG. 7B). The SWIR IR sensor element was an InGaAs type sensor equipped with an optical filter element that included at least two segments with high transmission for narrow wavelength ranges to allow only selected wavelength ranges to hit the sensor surface. In operation of the glucose sensing device, such as IR spectrometer 700B, as described above in more detail, at least one of the at least two wavelength ranges are chosen from the ranges with a maximum transmissivity around 1942 nm or 2098 nm, and the at least one other wavelength range is chosen from the ranges with a minimum transmissivity around 1890 nm or 2004 nm. Therefore, the temperature dependence of emission output for the potential wavelength ranges was measured for SWIR phosphor light emitting device formed in Example 9. FIG. 26 shows the variation of the light source emission power driven with direct current (150 mA drive current DC) in the range of human skin temperature for the proposed sensing wavelength ranges. In particular, emission power change, normalized to 37 C, was measured for four selected wavelength ranges: 1884-1896 nm (2671), 1998-2010 nm (2673), 1936-1948 nm (2675), and 2092-2104 nm (2677) at ambient temperature ranges of 28-44° C., which are the temperature ranges of human skin temperature). For the spectral intervals measured, each having a width of 12 nm, emission power changes of −0.08%/K to −0.11%/K were measured.

This disclosure is illustrative and not limiting. Further modifications will be apparent to one skilled in the art in light of this disclosure and are intended to fall within the scope of the appended claims.

1. A glucose measurement device comprising:
a light emitting device comprising a SWIR phosphor having emission wavelengths in the range of 1600-2200 nm, the SWIR phosphor comprising a structurally disordered garnet material, a sensitizer ion, and at least one rare earth emitter ion, wherein the at least one rare earth emitter ion comprises Tm(III); and
an infrared light detector arranged to detect the intensity of infrared light emitted by the light emitting device and reflected by a sample.

2. The glucose measurement device of claim 1, wherein the at least one rare earth emitter ion further comprises Ho(III).

3. The glucose measurement device of claim 1, wherein the sensitizer ion comprises Cr(III).

4. The glucose measurement device of claim 2, wherein the sensitizer ion comprises Cr(III).

5. The glucose measurement device of claim 1, wherein the SWIR phosphor comprises:
(Gd_{3-u-v-x-y-z}LuₓTm_{y}Ho_{z}ScᵥREᵤ)[Sc_{2-a-b-d-e}LuₐCr_{b}Ga_{d} Alₑ]{Ga_{3-c}Ale}O₁₂ with RE=La, Y, Yb, Nd, Er, Ce and 0<u<2, 0<v≤1, 0<x<1, 0<y≤0.5, 0<z≤0.05, 0<a≤1, 0<b≤0.3, 0<c≤3, 0<d≤1.8, 0<e≤1.8.

6. The glucose measurement device of claim 1, wherein the SWIR phosphor comprises Gd₂O₃- 0.0065 Ho₂O₃- 0.1 Tm₂O₃- 0.33 Sc₂O₃-0.12 Lu₂O₃- 0.8 Ga₂O₃- 0.04 Cr₂O₃-1.6 Al₂O₃.

7. The glucose measurement device of claim 1, wherein the SWIR phosphor is formed into a ceramic plate, the ceramic plate including (Al,Ga)₂O₃ as a minority phase.

8. The glucose measurement device of claim 1, wherein the light emitting device comprises an InGaN primary light source, the SWIR phosphor formed into a wavelength converting structure disposed on a light emitting face of the primary light source, the wavelength converting structure further comprising at least two alternating silica and niobia oxide layers disposed on a surface of the wavelength converting structure opposite the primary light source.

9. The glucose measurement device of claim 1, wherein the light emitting device comprises a light emitting surface, the light emitting surface having an area of 1 cm×1 cm or less.

10. The glucose measurement device of claim 1, wherein the light emitting device comprises an array of pcLEDs.

11. The glucose measurement device of claim 1, wherein the infrared light detector comprises a filter element, the filter element configured to selectively pass at least one of the wavelength ranges at a glucose infrared absorption maxima and a glucose infrared absorption minima.

12. The glucose measurement device of claim 1, wherein the infrared light detector comprises a filter element, the filter element configured to selectively pass at least one of the wavelength ranges 1942±0-12 nm, 2098±0-12 nm, 1890±0-12 nm, and 2004±0-12 nm.

13. The glucose measurement device of claim 1, wherein the light emitting device is configured to emit a SWIR spectral power output of >15 mW when driven at or near 150 mA.

14. The glucose measurement device of claim 1, wherein the light emitting device is configured to emit a spectrum that is thermally stable in the range of human skin temperatures.

15. The glucose measurement device of claim 1, wherein the light emitting device is configured to emit a spectrum that has less than a 0.1±0.02% /K linear intensity change at the glucose absorption spectrum maxima and minima over the temperature range 26° C. -56° C.

16. The glucose measurement device of claim 1, wherein the light emitting device is adjacent the infrared light detector, a light emitting surface of the light emitting device facing the sample, and a light receiving surface of the infrared light detector facing the sample.

17. A wearable device comprising the glucose measurement device of claim 1.

18. A method for determining glucose levels in a tissue sample comprising measuring reflection spectra from the skin sample at least one of the wavelengths within intervals 1942±0-12 nm, 2098±0-12 nm, 1890±0-12 nm, and 2004±0-12 nm using the glucose measurement device of claim 1.

19. The method for determining glucose levels of claim 18, wherein the glucose measurement device is a wearable device and the method is performed while the glucose measurement device is being worn by a user.

20. A glucose measurement device comprising:
a light emitting device comprising a SWIR phosphor having emission wavelengths in the range of 1600-2200 nm, the SWIR phosphor comprising a structurally disordered garnet material, a sensitizer ion, and at least one rare earth emitter ion, wherein the at least one rare earth emitter ion comprises Ho(III); and
an infrared light detector arranged to detect the intensity of infrared light emitted by the light emitting device and reflected by a sample.
